(19) United States
(12) Patent Application Publication
(10) Pub. No.: US 2022/0168452 A1
(43) Pub. Date: Jun. 2, 2022

(54) TISSUE SPACERS

(71) Applicant: Palette Life Sciences, Inc., Santa Barbara, CA (US)

(72) Inventor: Brent Travis GAY, Santa Barbara, CA (US)

(21) Appl. No.: 17/522,674

(22) Filed: Nov. 9, 2021

Related U.S. Application Data
(63) Continuation of application No. PCT/US2020/031056, filed on May 1, 2020.
(60) Provisional application No. 62/843,267, filed on May 3, 2019.

(51) Int. Cl. A61K 51/12; A61K 51/02; A61L 27/20

(52) U.S. Cl. CPC A61K 51/1213; A61L 27/20; A61K 51/025

(57) ABSTRACT

Provided herein are methods for decreasing the toxicity of advanced ablative cancer therapies on neighboring organs. The methods herein provide spacing between single or multiple tumor cites and immediate healthy organs while maintaining or increasing patient quality of life. Such toxicity isolation can be performed by inserting a spacer around the one or more tumor cites, which can be performed concurrently with fiducial marker placement.

### CROSS-REFERENCE

This application is a continuation of International Application No.: PCT/US2020/031056, filed May 1, 2020, which claims the benefit of U.S. Provisional Application No. 62/843,267, filed May 3, 2019, which are hereby incorporated by reference in their entirety herein.

### BACKGROUND

Generally, radiotherapy is considered as a palliative treatment option for severe or uncommon cases of melanoma. However there has recently been an increased demand for new systems and methods of melanoma management. Brachytherapy techniques also involve balloon or strut multicatheter brachytherapy, with the applicator being placed in the surgical cavity by the breast surgeon at the time of or shortly after the wide local excision.

### SUMMARY

The formulations and methods described herein comprise improved methods of radiotherapy. More specifically, the formulations and methods described herein comprise reducing a dose of radiotherapy to tissue proximate to the site of radiotherapy.

Also provided herein is use of a viscoelastic medium for the manufacture of a medicament. In some embodiments, more than 70% (v/v) of the particles are within the given size limits under physiological conditions, including man. Provided herein are particles of a viscoelastic medium, which are injectable gel particles having a size, when subjected to a physiological salt solution, in the range of from 1 to 5 mm. Sub-epidermal administration of an implant comprising gel particles made of a viscoelastic medium which are considerably larger than previously used in implants made of viscoelastic media are useful in avoiding migration and/or displacement of the implant, or part thereof, from the desired site of radiative protection. Moreover, the limited displacement of the implant in combination with the considerable particle size can facilitate easy removal of the implant, if desired. In an embodiment herein, said particle size is in the range of from 1 to 2.5 mm. In some embodiments, said size is in the range of from 2.5 to 5 mm. In embodiments herein, said viscoelastic medium is selected from the group consisting of polysaccharides and derivatives thereof. In some embodiments, said viscoelastic medium is selected from stabilized glycosaminoglycans and derivatives thereof. In some embodiments, said viscoelastic medium is selected from the group consisting of stabilized hyaluronic acid, stabilized chondroitin sulfate, stabilized heparin, and derivatives thereof. In some embodiments herein, said viscoelastic medium is selected from the group consisting of cross-linked hyaluronic acid and derivatives thereof. In some embodiments, the concentration of said viscoelastic medium in said gel particles, when subjected to a physiological salt solution, is in the range of from 5 to 100 mg/ml. In some embodiments, the particles herein are injectable through a 20 gauge or larger needle by application of a pressure of 15-50 N.

Further, provided herein is a method of producing injectable gel particles of a viscoelastic medium, comprising the steps of: (i) manufacturing a gel with a desired concentration of said viscoelastic medium; and (ii) mechanically disrupting said gel into gel particles having a size, when subjected to a physiological salt solution, in the range of from 1 to 5 mm.

Further, provided herein is a radiative protection implant comprising particles of a viscoelastic medium, wherein a major volume of said particles are injectable gel particles having a size, when subjected to a physiological salt solution, in the range of from 1 to 5 mm. In one embodiment of the implant, said size is in the range of from 1 to 2.5 mm. In other one embodiments of the implant, said size is in the range of from 2.5 to 5 mm.

Further, provided herein is a method of radiative protection of neighboring organs in a mammal, including man, comprising subepidermal administration at a site in said mammal where soft tissue radiative protection is desirable, of an implant comprising injectable gel particles of a viscoelastic medium, a major volume of said particles having a size, when subjected to a physiological salt solution, in the range of from 1 to 5 mm. In some embodiments, said administration is selected from the group consisting of subcutaneous administration, submuscular administration and supraperiostal administration. In some embodiments, said size is in the range of from 1 to 2.5 mm. In some embodiments, said site of radiative protection is selected from facial tissue and other tissues covered by exposed skin. In some embodiments, said size is in the range of from 2.5 to 5 mm. In some embodiments, said administration is a selected from the group consisting of single administration and multiple-layer administration.

Further, provided herein are injectable gel particles according to an embodiment herein for use as a medicament. Further, provided herein is an injectable radiative protection implant comprising injectable gel particles according to an embodiment herein for use as a medicament.

Further, provided herein is a method of using an injectable gel particles of a viscoelastic medium according to an embodiment herein. In some embodiments, the particles have an average size when subjected to a physiological salt solution, in the range of from 1 to 5 mm, for the manufacture of a medicament for therapeutic radiative protection in a mammal, including man, wherein said medicament is suitable for subepidermal administration according to an embodiment herein at a site in said mammal where therapeutic radiative protection is desirable.

Further provided herein are particles of a viscoelastic medium, which are injectable gel particles having a size, when subjected to a physiological salt solution, in the range of from 1 to 5 mm. The particles are useful in a radiative protection implant comprising particles of a viscoelastic medium, wherein a major volume of said particles are injectable gel particles having a specific size or range of sizes, when subjected to a physiological salt solution, in the range of from 1 to 5 mm. The implant, in turn, is useful in a method of radiative protection in a mammal, including man, comprising subepidermal administration at a site in said mammal where radiative protection is desirable, of an implant comprising injectable gel particles of a viscoelastic medium, a major volume of said particles having a size, when subjected to a physiological salt solution, in the range of from 1 to 5 mm.

Another aspect provided herein is a method of preventing or decreasing damage to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise hyaluronic acid or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, a dose of the radiotherapy contacting the tissue proximate to the site of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20%, about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15%, about 20%, about 30%, about 40%, about 50%, about 60%, about 70%, about 80%, or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of reducing a dose of radiotherapy to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise hyaluronic acid or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, the dose of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20%, about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15%, about 20%, about 30%, about 40%, about 50%, about 60%, about 70%, about 80%, or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of temporarily super-spacing a tissue proximate to a site of radiotherapy comprising injecting a formulation comprising cross-linked hyaluronic acid or derivatives thereof and an amount of degradable nanoparticles encapsulating hyaluronidase. In some embodiments, the amount of degradable nanoparticles encapsulating hyaluronidase is directly proportionate to a desired distance of super spacing relative to a desired time period of super spacing.

Another aspect provided herein is a method of treating cancer in subject suffering thereof comprising injecting a bioabsorbable viscoelastic medium in a blood vessel wherein the blood vessel is directly coupled to a tumor. In some embodiments, the viscoelastic medium comprises gel particles. In some embodiments, the gel particles comprise hyaluronic acid or derivatives thereof.

In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the hyaluronic acid in the spacer material is about 1 mg/ml to about 100 mg/ml. In some embodiments, the concentration of the hyaluronic acid in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the hyaluronic acid in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the hyaluronic acid in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the hyaluronic acid in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, blood flow to the tumor is prevented or decreased. In some embodiments, migration of the viscoelastic medium is prevented or decreased. In some embodiments, the method further comprises an administration of hyaluronidase at the site of radiotherapy.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

In some embodiments, the method further comprises excising the remaining tumor cells from the subject.

Another aspect provided herein is a formulation comprising cross-linked hyaluronic acid and a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, and ioversol. In some embodiments, the formulation is used as a fiducial marker.

Another aspect provided herein is a method of preventing or decreasing damage to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise polyethylene glycol or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, a dose of the radiotherapy contacting the tissue proximate to the site of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20%, about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15%, about 20%, about 30%, about 40%, about 50%, about 60%, about 70%, about 80%, or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of reducing a dose of radiotherapy to a tissue proximate to a site of a radiotherapy in a subject undergoing the radiotherapy comprising an injection of a bioabsorbable viscoelastic medium at the site of the radiotherapy. In some embodiments, the viscoelastic medium comprises gel particles. In some embodiments, the gel particles comprise polyethylene glycol or derivatives thereof.

In some embodiments, the injection displaces the tissue by a distance of about 0.1 cm to about 10 cm. In some embodiments, the injection displaces the tissue by a distance of about 0.1 cm to about 0.2 cm, about 0.1 cm to about 0.5 cm, about 0.1 cm to about 1 cm, about 0.1 cm to about 2 cm, about 0.1 cm to about 3 cm, about 0.1 cm to about 4 cm, about 0.1 cm to about 5 cm, about 0.1 cm to about 6 cm, about 0.1 cm to about 7 cm, about 0.1 cm to about 8 cm, about 0.1 cm to about 10 cm, about 0.2 cm to about 0.5 cm, about 0.2 cm to about 1 cm, about 0.2 cm to about 2 cm, about 0.2 cm to about 3 cm, about 0.2 cm to about 4 cm, about 0.2 cm to about 5 cm, about 0.2 cm to about 6 cm, about 0.2 cm to about 7 cm, about 0.2 cm to about 8 cm, about 0.2 cm to about 10 cm, about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 10 cm, about 4 cm to about 5 cm, about 4 cm to about 6 cm, about 4 cm to about 7 cm, about 4 cm to about 8 cm, about 4 cm to about 10 cm, about 5 cm to about 6 cm, about 5 cm to about 7 cm, about 5 cm to about 8 cm, about 5 cm to about 10 cm, about 6 cm to about 7 cm, about 6 cm to about 8 cm, about 6 cm to about 10 cm, about 7 cm to about 8 cm, about 7 cm to about 10 cm, or about 8 cm to about 10 cm. In some embodiments, the injection displaces the tissue by a distance of about 0.1 cm, about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection displaces the tissue by a distance of at least about 0.1 cm, about 0.2 cm, about 0.5 cm, about 1 cm, about 2 cm, about 3 cm, about 4 cm, about 5 cm, about 6 cm, about 7 cm, or about 8 cm. In some embodiments, the injection displaces the tissue by a distance of at most about 0.2 cm, about 0.5 cm, about 1 cm, about 2 cm, about 3 cm, about 4 cm, about 5 cm, about 6 cm, about 7 cm, about 8 cm, or about 10 cm. In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the injection is subcutaneous or subepidermal. In some embodiments, migration of the viscoelastic medium is prevented or decreased. In some embodiments, the viscoelastic medium further comprises nanoparticles. In some embodiments, the nanoparticles comprise a precious metal. In some embodiments, the dose of radiotherapy is reduced by about 10% to about 80%. In some embodiments, the site of the radiotherapy is selected from a group consisting of the subject's breast, head & neck, cervix, vagina, base of spine, skin, pancreas, liver, or lung. In some embodiments, the method further comprises an administration of hyaluronidase at the site of radiotherapy.

In some embodiments, the volume of the viscoelastic medium at the site of radiotherapy is reduced by about 1% to about 95%. In some embodiments, the volume of the viscoelastic medium at the site of radiotherapy is reduced by about 1% to about 5%, about 1% to about 10%, about 1% to about 15%, about 1% to about 20%, about 1% to about 30%, about 1% to about 40%, about 1% to about 50%, about 1% to about 60%, about 1% to about 70%, about 1% to about 80%, about 1% to about 95%, about 5% to about 10%, about 5% to about 15%, about 5% to about 20%, about 5% to about 30%, about 5% to about 40%, about 5% to about 50%, about 5% to about 60%, about 5% to about 70%, about 5% to about 80%, about 5% to about 95%, about 10% to about 15%, about 10% to about 20%, about 10% to about 30%, about 10% to about 40%, about 10% to about 50%, about 10% to about 60%, about 10% to about 70%, about 10% to about 80%, about 10% to about 95%, about 15% to about 20%, about 15% to about 30%, about 15% to about 40%, about 15% to about 50%, about 15% to about 60%, about 15% to about 70%, about 15% to about 80%, about 15% to about 95%, about 20% to about 30%, about 20% to about 40%, about 20% to about 50%, about 20% to about 60%, about 20% to about 70%, about 20% to about 80%, about 20% to about 95%, about 30% to about 40%, about 30% to about 50%, about 30% to about 60%, about 30% to about 70%, about 30% to about 80%, about 30% to about 95%, about 40% to about 50%, about 40% to about 60%, about 40% to about 70%, about 40% to about 80%, about 40% to about 95%, about 50% to about 60%, about 50% to about 70%, about 50% to about 80%, about 50% to about 95%, about 60% to about 70%, about 60% to about 80%, about 60% to about 95%, about 70% to about 80%, about 70% to about 95%, or about 80% to about 95%. In some embodiments, the volume of the viscoelastic medium at the site of radiotherapy is reduced by about 1%, about 5%, about 10%, about 15%, about 20%, about 30%, about 40%, about 50%, about 60%, about 70%, about 80%, or about 95%. In some embodiments, the volume of the viscoelastic medium at the site of radiotherapy is reduced by at least about 1%, about 5%, about 10%, about 15%, about 20%, about 30%, about 40%, about 50%, about 60%, about 70%, or about 80%. In some embodiments, the volume of the viscoelastic medium at the site of radiotherapy is reduced by at most about 5%, about 10%, about 15%, about 20%, about 30%, about 40%, about 50%, about 60%, about 70%, about 80%, or about 95%.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

Another aspect provided herein is a method of temporarily super-spacing a tissue proximate to a site of radiotherapy comprising injecting a formulation comprising cross-linked polyethylene glycol or derivatives thereof and an amount of degradable nanoparticles encapsulating hyaluronidase. In some embodiments, the amount of degradable nanoparticles encapsulating hyaluronidase is directly proportionate to a desired distance of super spacing relative to a desired time period of super spacing.

Another aspect provided herein is a method of treating cancer in subject suffering thereof comprising injecting a bioabsorbable viscoelastic medium in a blood vessel wherein the blood vessel is directly coupled to a tumor. In some embodiments, the viscoelastic medium comprises gel particles. In some embodiments, the gel particles comprise polyethylene glycol or derivatives thereof.

In some embodiments, the injection comprises a volume of about 1 ml to about 50 ml. In some embodiments, the injection comprises a volume of about 1 ml to about 2 ml, about 1 ml to about 5 ml, about 1 ml to about 10 ml, about 1 ml to about 15 ml, about 1 ml to about 20 ml, about 1 ml to about 25 ml, about 1 ml to about 30 ml, about 1 ml to about 35 ml, about 1 ml to about 40 ml, about 1 ml to about 45 ml, about 1 ml to about 50 ml, about 2 ml to about 5 ml, about 2 ml to about 10 ml, about 2 ml to about 15 ml, about 2 ml to about 20 ml, about 2 ml to about 25 ml, about 2 ml to about 30 ml, about 2 ml to about 35 ml, about 2 ml to about 40 ml, about 2 ml to about 45 ml, about 2 ml to about 50 ml, about 5 ml to about 10 ml, about 5 ml to about 15 ml, about 5 ml to about 20 ml, about 5 ml to about 25 ml, about 5 ml to about 30 ml, about 5 ml to about 35 ml, about 5 ml to about 40 ml, about 5 ml to about 45 ml, about 5 ml to about 50 ml, about 10 ml to about 15 ml, about 10 ml to about 20 ml, about 10 ml to about 25 ml, about 10 ml to about 30 ml, about 10 ml to about 35 ml, about 10 ml to about 40 ml, about 10 ml to about 45 ml, about 10 ml to about 50 ml, about 15 ml to about 20 ml, about 15 ml to about 25 ml, about 15 ml to about 30 ml, about 15 ml to about 35 ml, about 15 ml to about 40 ml, about 15 ml to about 45 ml, about 15 ml to about 50 ml, about 20 ml to about 25 ml, about 20 ml to about 30 ml, about 20 ml to about 35 ml, about 20 ml to about 40 ml, about 20 ml to about 45 ml, about 20 ml to about 50 ml, about 25 ml to about 30 ml, about 25 ml to about 35 ml, about 25 ml to about 40 ml, about 25 ml to about 45 ml, about 25 ml to about 50 ml, about 30 ml to about 35 ml, about 30 ml to about 40 ml, about 30 ml to about 45 ml, about 30 ml to about 50 ml, about 35 ml to about 40 ml, about 35 ml to about 45 ml, about 35 ml to about 50 ml, about 40 ml to about 45 ml, about 40 ml to about 50 ml, or about 45 ml to about 50 ml. In some embodiments, the injection comprises a volume of about 1 ml, about 2 ml, about 5 ml, about 10 ml, about 15 ml, about 20 ml, about 25 ml, about 30 ml, about 35 ml, about 40 ml, about 45 ml, or about 50 ml. In some embodiments, the injection comprises a volume of at least about 1 ml, about 2 ml, about 5 ml, about 10 ml, about 15 ml, about 20 ml, about 25 ml, about 30 ml, about 35 ml, about 40 ml, or about 45 ml. In some embodiments, the injection comprises a volume of at most about 2 ml, about 5 ml, about 10 ml, about 15 ml, about 20 ml, about 25 ml, about 30 ml, about 35 ml, about 40 ml, about 45 ml, or about 50 ml.

In some embodiments, the injection is performed by a needle having a gauge of about 10 to about 26. In some embodiments, the injection is performed by a needle having a gauge of about 10 to about 11, about 10 to about 12, about 10 to about 13, about 10 to about 14, about 10 to about 15, about 10 to about 16, about 10 to about 18, about 10 to about 20, about 10 to about 22, about 10 to about 24, about 10 to about 26, about 11 to about 12, about 11 to about 13, about 11 to about 14, about 11 to about 15, about 11 to about 16, about 11 to about 18, about 11 to about 20, about 11 to about 22, about 11 to about 24, about 11 to about 26, about 12 to about 13, about 12 to about 14, about 12 to about 15, about 12 to about 16, about 12 to about 18, about 12 to about 20, about 12 to about 22, about 12 to about 24, about 12 to about 26, about 13 to about 14, about 13 to about 15, about 13 to about 16, about 13 to about 18, about 13 to about 20, about 13 to about 22, about 13 to about 24, about 13 to about 26, about 14 to about 15, about 14 to about 16, about 14 to about 18, about 14 to about 20, about 14 to about 22, about 14 to about 24, about 14 to about 26, about 15 to about 16, about 15 to about 18, about 15 to about 20, about 15 to about 22, about 15 to about 24, about 15 to about 26, about 16 to about 18, about 16 to about 20, about 16 to about 22, about 16 to about 24, about 16 to about 26, about 18 to about 20, about 18 to about 22, about 18 to about 24, about 18 to about 26, about 20 to about 22, about 20 to about 24, about 20 to about 26, about 22 to about 24, about 22 to about 26, or about 24 to about 26. In some embodiments, the injection is performed by a needle having a gauge of about 10, about 11, about 12, about 13, about 14, about 15, about 16, about 18, about 20, about 22, about 24, or about 26. In some embodiments, the injection is performed by a needle having a gauge of at least about 10, about 11, about 12, about 13, about 14, about 15, about 16, about 18, about 20, about 22, or about 24. In some embodiments, the injection is performed by a needle having a gauge of at most about 11, about 12, about 13, about 14, about 15, about 16, about 18, about 20, about 22, about 24, or about 26.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, blood flow to the tumor is prevented or decreased. In some embodiments, migration of the viscoelastic medium is prevented or decreased. In some embodiments, the method further comprises an administration of hyaluronidase at the site of radiotherapy.

In some embodiments, the administration of hyaluronidase occurs at a time after injection of the bioabsorbable viscoelastic medium of about 0.1 hours to about 95 hours. In some embodiments, the administration of hyaluronidase occurs at a time after injection of the bioabsorbable viscoelastic medium of about 0.1 hours to about 0.5 hours, about 0.1 hours to about 1 hour, about 0.1 hours to about 2 hours, about 0.1 hours to about 4 hours, about 0.1 hours to about 6 hours, about 0.1 hours to about 8 hours, about 0.1 hours to about 10 hours, about 0.1 hours to about 14 hours, about 0.1 hours to about 18 hours, about 0.1 hours to about 24 hours, about 0.1 hours to about 95 hours, about 0.5 hours to about 1 hour, about 0.5 hours to about 2 hours, about 0.5 hours to about 4 hours, about 0.5 hours to about 6 hours, about 0.5 hours to about 8 hours, about 0.5 hours to about 10 hours, about 0.5 hours to about 14 hours, about 0.5 hours to about 18 hours, about 0.5 hours to about 24 hours, about 0.5 hours to about 95 hours, about 1 hour to about 2 hours, about 1 hour to about 4 hours, about 1 hour to about 6 hours, about 1 hour to about 8 hours, about 1 hour to about 10 hours, about 1 hour to about 14 hours, about 1 hour to about 18 hours, about 1 hour to about 24 hours, about 1 hour to about 95 hours, about 2 hours to about 4 hours, about 2 hours to about 6 hours, about 2 hours to about 8 hours, about 2 hours to about 10 hours, about 2 hours to about 14 hours, about 2 hours to about 18 hours, about 2 hours to about 24 hours, about 2 hours to about 95 hours, about 4 hours to about 6 hours, about 4 hours to about 8 hours, about 4 hours to about 10 hours, about 4 hours to about 14 hours, about 4 hours to about 18 hours, about 4 hours to about 24 hours, about 4 hours to about 95 hours, about 6 hours to about 8 hours, about 6 hours to about 10 hours, about 6 hours to about 14 hours, about 6 hours to about 18 hours, about 6 hours to about 24 hours, about 6 hours to about 95 hours, about 8 hours to about 10 hours, about 8 hours to about 14 hours, about 8 hours to about 18 hours, about 8 hours to about 24 hours, about 8 hours to about 95 hours, about 10 hours to about 14 hours, about 10 hours to about 18 hours, about 10 hours to about 24 hours, about 10 hours to about 95 hours, about 14 hours to about 18 hours, about 14 hours to about 24 hours, about 14 hours to about 95 hours, about 18 hours to about 24 hours, about 18 hours to about 95 hours, or about 24 hours to about 95 hours. In some embodiments, the administration of hyaluronidase occurs at a time after injection of the bioabsorbable viscoelastic medium of about 0.1 hours, about 0.5 hours, about 1 hour, about 2 hours, about 4 hours, about 6 hours, about 8 hours, about 10 hours, about 14 hours, about 18 hours, about 24 hours, or about 95 hours. In some embodiments, the administration of hyaluronidase occurs at a time after injection of the bioabsorbable viscoelastic medium of at least about 0.1 hours, about 0.5 hours, about 1 hour, about 2 hours, about 4 hours, about 6 hours, about 8 hours, about 10 hours, about 14 hours, about 18 hours, or about 24 hours. In some embodiments, the administration of hyaluronidase occurs at a time after injection of the bioabsorbable viscoelastic medium of at most about 0.5 hours, about 1 hour, about 2 hours, about 4 hours, about 6 hours, about 8 hours, about 10 hours, about 14 hours, about 18 hours, about 24 hours, or about 95 hours.

In some embodiments, the method further comprises excising the remaining tumor cells from the subject.

Another aspect provided herein is a formulation comprising cross-linked polyethylene glycol and a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, and ioversol. In some embodiments, the formulation is used as a fiducial marker.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is an exemplary image of an injected area of a mastectomy sample; according to an embodiment herein;

FIG. 1B is an exemplary image of injecting a spacer into a mastectomy sample using ultrasound guidance; according to an embodiment herein;

FIG. 2A is an ultrasound exemplary image of gland and adipose tissue within a mastectomy sample; according to an embodiment herein;

FIG. 2B is an ultrasound exemplary image of a spacer, gland tissue, and adipose tissue within a mastectomy sample; according to an embodiment herein;

FIG. 3A is an exemplary computed tomography (CT) scan of a hyaluronic acid (HA) spacer within a mastectomy sample; according to an embodiment herein;

FIG. 3B is an exemplary ultrasound image of a HA spacer within a mastectomy sample; according to an embodiment herein;

FIG. 3C is an exemplary CT scan of a polyethylene glycol (PEG) spacer within a mastectomy sample; according to an embodiment herein;

FIG. 3D is an exemplary CT scan of a PGA spacer within a mastectomy sample; according to an embodiment herein;

FIG. 4A is an exemplary image of an image of a simulated permanent breast seed implant (PBSI) brachytherapy planning of a hyaluronic acid (HA) spacer within a mastectomy sample; according to an embodiment herein;

FIG. 4B is an exemplary ultrasound image of a HA spacer within a mastectomy sample; according to an embodiment herein;

FIG. 5A is an exemplary image of a computed tomography scan before hydrogel spacer injection between the head of the pancreas and duodenum, according to an embodiment herein;

FIG. 5B is an exemplary image of a computed tomography scan after hydrogel spacer injection between the head of the pancreas and duodenum, according to an embodiment herein;

FIG. 5C is an exemplary image of a gross histologic specimen after hydrogel spacer injection between the head of the pancreas and duodenum, according to an embodiment herein;

FIG. 5D is an exemplary image of a computed tomography scan before a laparotomy hydrogel spacer injection between the head of the pancreas and duodenum, according to an embodiment herein;

FIG. 5E is an exemplary image of a computed tomography scan after a laparotomy hydrogel spacer injection between the head of the pancreas and duodenum, according to an embodiment herein;

FIG. 5F is an exemplary image of a gross histologic specimen after a laparotomy hydrogel spacer injection between the head of the pancreas and duodenum, according to an embodiment herein;

FIG. 5G is an exemplary image of a computed tomography scan before an endoscopically hydrogel spacer injection between the head of the pancreas and duodenum, according to an embodiment herein;

FIG. 5H is an exemplary image of a computed tomography scan after an endoscopically hydrogel spacer injection between the head of the pancreas and duodenum, according to an embodiment herein;

FIG. 5I is an exemplary image of a gross histologic specimen after an endoscopically hydrogel spacer injection between the head of the pancreas and duodenum, according to an embodiment herein;

FIG. 6A is a first exemplary image of a formalin-fixed, paraffin-embedded section after hematoxylin and eosin staining, according to an embodiment herein;

FIG. 6B is a second exemplary image of a formalin-fixed, paraffin-embedded section after hematoxylin and eosin staining, according to an embodiment herein;

FIG. 6C is a first exemplary high magnification image of a formalin-fixed, paraffin-embedded section after hematoxylin and eosin staining, according to an embodiment herein;

FIG. 6D is a third exemplary image of a formalin-fixed, paraffin-embedded section after hematoxylin and eosin staining, according to an embodiment herein;

FIG. 6E is a second exemplary high magnification image of a formalin-fixed, paraffin-embedded section after hematoxylin and eosin staining, according to an embodiment herein;

FIG. 7A is a first exemplary stereotactic body radiation therapy plan before hydrogel spacer placement, according to an embodiment herein;

FIG. 7B is a first exemplary stereotactic body radiation therapy plan after hydrogel spacer placement, according to an embodiment herein;

FIG. 7C is a second exemplary stereotactic body radiation therapy plan before hydrogel spacer placement, according to an embodiment herein;

FIG. 7D is a second exemplary stereotactic body radiation therapy plan after hydrogel spacer placement, according to an embodiment herein;

FIG. 8A is an exemplary baseline image of a computed tomography scan and stereotactic body radiation therapy plan of the duodenum, according to an embodiment herein;

FIG. 8B is an exemplary image of a computed tomography scan and stereotactic body radiation therapy plan of the duodenum with a 2 mm spacing, according to an embodiment herein;

FIG. 8C is an exemplary image of a computed tomography scan and stereotactic body radiation therapy plan of the duodenum with a 3 mm spacing, according to an embodiment herein;

FIG. 8D is an exemplary image of a computed tomography scan and stereotactic body radiation therapy plan of the duodenum with a 5 mm spacing, according to an embodiment herein;

FIG. 8E is an exemplary image of a computed tomography scan and stereotactic body radiation therapy plan of the duodenum with a 8 mm spacing, according to an embodiment herein;

FIG. 8F is an exemplary image of a computed tomography scan and stereotactic body radiation therapy plan of the duodenum with a 15 mm spacing, according to an embodiment herein;

FIG. 9 shows an MRI scan of bladder markings, a CT scan of liver markings, an MRI of liver markings and an MRI of cervical markings, according to an embodiment herein;

FIG. 10A shows an MRI scan of a submandibular tumor before treatment, according to an embodiment herein;

FIG. 10B shows an MRI scan of a submandibular tumor with distance measurements of about 1 cm, according to an embodiment herein;

FIG. 10C shows an MRI 6 months after the tumor was removed, according to an embodiment herein;

FIG. 11A shows an image of an applicator needle inserted from the left side of the neck, according to an embodiment herein;

FIG. 11B shows an image of the location of a 20 Gy single dose of radiation, according to an embodiment herein;

FIG. 12 shows an MRI image of a paravertebral dosing approach, according to an embodiment herein;

FIG. 13A shows an illustration of a reconstructed rectum and prostate before a brachytherapy radiation, according to an embodiment herein;

FIG. 13B shows an illustration of a reconstructed rectum and prostate after the initial brachytherapy radiation, according to an embodiment herein;

FIG. 14A shows an illustration radiation levels before a brachytherapy radiation with the rectum and prostate being separated by more than 25 mm, according to an embodiment herein;

FIG. 14B shows an illustration radiation levels after a brachytherapy radiation with the rectum and prostate being separated by more than 25 mm, according to an embodiment herein;

FIG. 14C shows an MRI scan of the prostrate and the rectum four hours after injection, according to an embodiment herein;

FIG. 15A shows an X-ray computed tomography image before radiotherapy, according to an embodiment herein;

FIG. 15B shows an X-ray computed tomography image before and external beam radiotherapy treatment plan radiotherapy, according to an embodiment herein;

FIG. 16A shows an ultrasound image and power Doppler image showing the planned route for needle insertion, according to an embodiment herein;

FIG. 16B shows an ultrasound image and power Doppler image showing Brachytherapy dose distribution and inserted brachytherapy needle, according to an embodiment herein;

FIG. 16C shows an ultrasound image after the skin is raised 7 mm, according to an embodiment herein;

FIG. 17A shows an X-ray computed tomography and brachytherapy dose distribution, according to an embodiment herein;

FIG. 17B shows an X-ray computed tomography and images of the second lesion according to an embodiment herein; and

FIG. 17C shows an X-ray computed tomography with no tumor recurrence at 1 year after treatment, according to an embodiment herein.

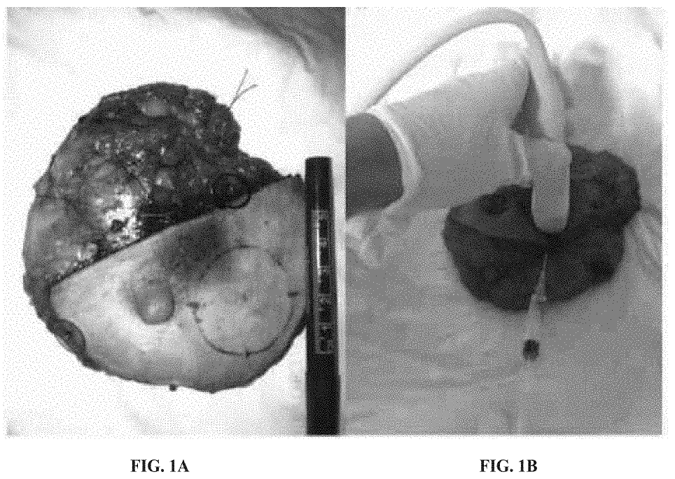

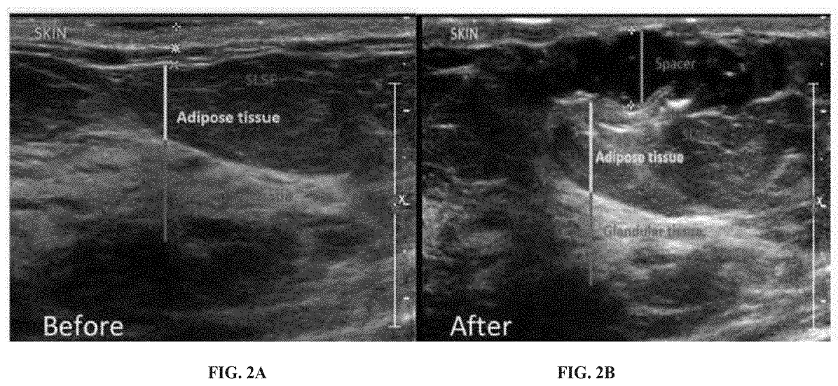

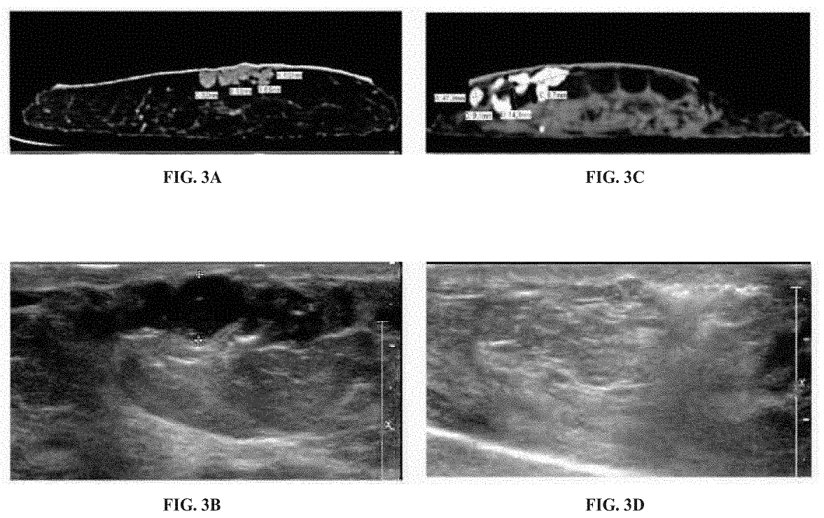

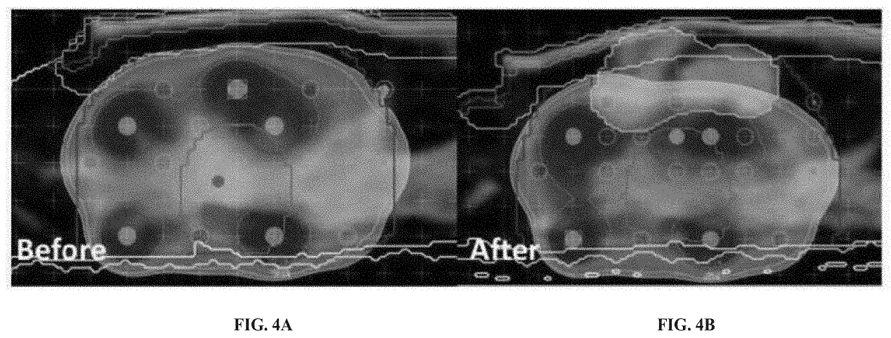

### DETAILED DESCRIPTION

Provided herein are methods for decreasing the toxicity of advanced ablative cancer therapies on neighboring organs. The methods herein provide spacing between single or multiple tumor cites and immediate healthy organs while maintaining or increasing patient quality of life. Such toxicity isolation can be performed by inserting a spacer around the one or more tumor cites, which can be performed concurrently with fiducial marker placement.

### Subcutaneous Spacer Materials

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation.

In some embodiments, the implant comprises particles of one or more viscoelastic media dispersed in a physiological salt buffer, a suitable physiological salt solvent, or both. In some embodiments, the implant further comprises other additives, such as local anesthetics, anti-inflammatory drugs, antibiotics and supportive medications (e.g. bone growth factors or cells). In some embodiments, there may also be included a viscoelastic medium which may be formed of same material as the particles or a different material than the particles. In some embodiments, the viscoelastic medium is not present as particles.

Viscoelastic media according to embodiments can herein include gels, dispersions, solutions, suspensions, slurries and mixtures thereof. In some embodiments, the medium is present as a dispersion of gel or gel-like particles. The viscoelastic medium provided herein can be more resistant to biodegradation in vivo than natural hyaluronic acid. The prolonged presence of the stable viscoelastic substance is advantageous for the patient, since the time between treatments is increased. The viscoelastic media herein can be biocompatible, sterile, and present as particles.

Advantageously, the viscoelastic media herein are stable within, but can be impermanent under, physiological conditions. In some embodiments, about 70% to about 90%, of the viscoelastic medium remains for at least two weeks in vivo. In some embodiments, at least 70%, of the viscoelastic medium remains for at about two weeks and two years in vivo. In some embodiments, at least 90%, of the viscoelastic medium remains for at about two weeks and two years in vivo. The viscoelastic medium can degrade automatically after five years or more in vivo.

Viscoelastic media include, without being limited thereto, polysaccharides and derivatives thereof. Suitable viscoelastic media include stabilized starch and derivatives thereof. Suitable viscoelastic media can also be selected from stabilized glycosaminoglycans and derivatives thereof, such as stabilized hyaluronic acid, stabilized chondroitin sulfate, stabilized heparin, and derivatives thereof. Suitable viscoelastic media also include stabilized dextran and derivatives thereof, such as dextranomer. In some embodiments, the dextronamer (Dx) has a molecular weight of about 40 kDa to about 70 kDa. In some embodiments, the Dx has a molecular weight of at most about 70 kDa. In some embodiments, the Dx has a molecular weight of at least about 40 kDa. In one example the viscoelastic media comprises hyaluronic acid and Dx. In another example the viscoelastic media comprises non-animal stabilized hyaluronic acid (NASHA)/Dx gel with de. In some embodiments, the non/Dx gel has a sufficiently low viscosity such that it can be injected through a syringe by finger pressure alone. In some embodiments, the NASHA/Dx gel has a sufficiently high viscosity to avoid leakage from the injection site. In some embodiments, the NASHA/Dx gel has a long degradation time which enables and stabilizes the natural formation of connective tissue at the site of the implant. In some embodiments, the viscoelastic medium further comprises carbon-coated zirconium beads, calcium hydroxylapatite, or both. In some embodiments, the viscoelastic medium is cross-linked hyaluronic acid, or a derivatives thereof. An example of a viscoelastic medium is NASHA. One type of suitable cross-linked hyaluronic acid is obtainable by cross-linking of hyaluronic acid. The viscoelastic medium may also be a combination of two or more of the suitable viscoelastic media listed herein or otherwise known to the art. The viscoelastic medium may be of non-animal origin.

In some embodiments, the viscoelastic medium comprises a hydrogel. In some embodiments, the hydrogel is formed from natural, synthetic, or biosynthetic polymers. In some embodiments, the natural polymer comprises glycosminoglycans, polysaccharides, proteins, or any combination thereof. In some embodiments, the glycosaminoglycan is dermatan sulfate, hyaluronic acid, chondroitin sulfate, chitin, heparin, keratan sulfate, keratosulfate, or any combination thereof. In some embodiments, the hydrogel comprises an acidic carboxy polymer, an acrylic acid-based polymer, a polyacrylamide, a starch graft copolymer, an acrylate polymer, or any combination thereof. In some embodiments, the hydrogel comprises allylpentaerythritol, polyacrylic acid, ester cross-linked polyglucan, or any combination thereof.

In some embodiments, the viscoelastic media is hydrophilic

The size of the gel particles can depend upon the ionic strength of the buffer, the solution, carrier, or any combination thereof that is included in and/or surrounding the gel particles. As such, given particle sizes can assume physiological conditions, particularly isotonic conditions. In some embodiments, the gel particles contain and are dispersed in a physiological salt solution. In some embodiments, the gel particles are temporarily brought to different sizes by subjecting the gel particles to a solution of another tonicity. Particle sizes within the given ranges under physiological conditions when implanted subepidermally in the body or when subjected to a physiological, or isotonic, salt solution (i.e. a solution with the same tonicity as the relevant biological fluids, such as an isoosmotic with serum).

In some embodiments, the particles have a specific tuned size. The size of the particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size. Particle size may be determined in any suitable way, such as by laser diffraction, microscopy, or filtration, etc. In some embodiments, the specific shape of the gel particles is not critical. The size of a spherical particle can equal its diameter. The size may be measured as an average size, a median size, a maximum size, or a minimum size.

In some embodiments, the particles have a size in the range of from 1 to 2.5 mm, such as from 1.5 to 2 mm, in the presence of a physiological salt solution. In some embodiments, the particles have a size in the range of from 2.5 to 5 mm, such as from 3 to 4 mm, in the presence of a physiological salt solution. At least 50% (v/v) of the particles can have a size of at least about 1 mm. At least 50% (v/v) of the particles can have a size of about 1-5 mm in the presence of a physiological salt solution. In some embodiments, more than 70% (v/v) of the particles are within the given size limits under physiological conditions. In some embodiments, more than 90% (v/v) of the particles are within the given size limits under physiological conditions. Administration of the implant employing the method according to an embodiment herein prevents or diminishes migration and/or displacement of the implant, which comprises or consists of the 1-5 mm large particles under physiological conditions. Large particles can exhibit less in vitro migration and can be more easily removed. In some embodiments, the viscoelastic medium is not present as particles of a size smaller than 0.1 mm. In some embodiments, the Dx is composed of microspheres. In some embodiments, the microspheres have a diameter of about 80 μm to about 250 μm. In some embodiments, the microspheres have a diameter of at least about 80 μm. In some embodiments, the microspheres have a diameter of at most about 250 μm. In some embodiments, the Dx is composed of microspheres. In some embodiments, the microspheres have a diameter of about 80 μm to about 250 μm. In some embodiments, the microspheres have a diameter of at least about 80 μm. In some embodiments, the microspheres have a diameter of at most about 250 μm

In some embodiments, the particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. Harder particles can be less viscoelastic and exhibit a longer half-life in vivo than softer particles. The particles herein should retain enough viscoelastic properties that they can be safely injected. In some embodiments, the implant comprises both soft gel particles and harder gel particles. The soft and hard gel particles may be made of the same or different viscoelastic media. The resulting mixture of gel particles combines desirable properties of softness/hardness for use in radiative protection and long durability in vivo.

### Methods of Forming a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation.

Provided herein are methods of forming a spacer material comprising forming an aqueous solution comprising: a water soluble cross-linkable polysaccharide; initiating a cross-linking of the polysaccharide in the presence of a polyfunctional cross-linking agent; sterically hindering the cross-linking reaction from terminating before gelation occurs to generate activated polysaccharide; and reintroducing the sterically unhindered conditions for the activated polysaccharide to continue the cross-linking thereof up to a viscoelastic gel. In some embodiments, the initial cross-linking reaction in the presence of a polyfunctional cross-linking agent can be performed at varying pH values, primarily depending on whether ether or ester reactions should be promoted.

The cross-linking agent can be any previously known cross-linking agent useful in connection with polysaccharides that are biocompatible. However, the cross-linking agent is comprises: aldehydes, epoxides, polyaziridyl compounds, glycidyl ethers, divinylsulfones, or any combination thereof. Glycidyl ethers represent an group, of which 1,4-butanediol diglycidyl ether can be advantageous. In some embodiments, the spacer material comprises a hydrogel comprising a glycosaminoglycan that is extracted from a natural source that is purified and derivatized. In some embodiments, the glycosaminoglycan is synthetically produced or synthesized by modified microorganisms such as bacteria. In some embodiments, the glycosaminoglycan is modified synthetically from a naturally soluble state to a partially soluble or water swellable or hydrogel state.

A suitable way of obtaining a desired particle size involves producing a gel made of cross-linked hyaluronic acid at a desired concentration and subjecting the gel to physical disruption, such as mincing, mashing or allowing the gel to pass through a filter with suitable particle size. The resulting gel particles are dispersed in a physiological salt solution, resulting in a gel dispersion or slurry with particles of desired size. The size of the particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size.

In some embodiments, the particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. By varying the hyaluronic acid concentrations to, for example, 20, 25, 40, 50 and 100 mg/ml gel particles of varying hardness can be obtained. Harder particles can be less viscoelastic and exhibit a longer half-life in vivo than softer particles. The particles herein should retain enough viscoelastic properties that they can be safely injected.

In some embodiments, the implant comprises both soft gel particles and harder gel particles. The soft and hard gel particles may be made of the same or different viscoelastic media. The resulting mixture of gel particles combines desirable properties of softness/hardness for use in radiative protection and long durability in vivo. In one embodiment the soft gel particles comprise 15-22 mg/ml of the cross-linked hyaluronic acid, and the hard gel particles comprise 22-30 mg/ml of the cross-linked hyaluronic acid.

When the injectable medium is a hyaluronic acid medium, the hyaluronic acid concentration can be at least about 5 mg/ml. In some embodiments, the hyaluronic acid concentration is about 5 mg/ml to about 100 mg/ml. In some embodiments, the hyaluronic acid concentration is about 10 to about 50 mg/ml. In some embodiments, the hyaluronic acid concentration is about 20 mg/ml. The cross-linked hyaluronic acid can be present as particles or beads of any form.

In some embodiments, the method further comprises adding an image enhancement agent described herein to the spacer material. In some embodiments, the method further comprises mixing in an image enhancement agent described herein to the spacer material.

### Methods of Injecting a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising hyaluronic acid particles. The particle size and concentration of the hyaluronic acid within the spacer material can be tuned to exhibit a hardness, density, or both to enable consistent and uniform injection and cavity formation. Further, a specific needle size can be used to deliver the spacer material to its intended in vivo location based on the particle size, hardness, density, and concentration of the hyaluronic acid.

Provided herein is a method of injecting a spacing material. The spacing material can comprise a viscoelastic medium for therapeutic radiative protection in a mammal, including man. The spacing material can suitable for subepidermal administration at a site in said mammal where therapeutic soft tissue protection is required from radiation or other toxic sources. In particular, the particles are suitable for administration to tissues covered by publicly exposed skin, such as facial tissue, as the particles do not cause bruises or other discolorations. The particles herein are suitable for administration into deep subcutaneous or to submuscular/supraperiostal tissue, optionally in more than one layer. Deep subcutaneous or submuscular/supraperiostal administration can further prevent or diminished migration of the particles away from the desired site.

The spacing material can be administered by injection under the epidermis in any suitable way. By way of example, a dermal incision can be made with a scalpel or a sharp injection needle to facilitate transdermal insertion of a larger cannula for administration of the implant at the desired site.

The implant, consisting of particles of a viscoelastic medium and optionally other suitable ingredients, may be administered as a single aliquot or as layers of multiple aliquots. Optionally, the viscoelastic medium may be replaced, refilled or replenished by a subsequent injection of the same or another viscoelastic medium. The injected volume is determined by the size of the desired cavity.

In some embodiments, a volume of the spacer material that is injected is about 1 ml to about 500 ml. In some embodiments, a volume of the spacer material that is injected is about 1 ml to about 5 ml, about 1 ml to about 10 ml, about 1 ml to about 25 ml, about 1 ml to about 50 ml, about 1 ml to about 100 ml, about 1 ml to about 150 ml, about 1 ml to about 200 ml, about 1 ml to about 250 ml, about 1 ml to about 300 ml, about 1 ml to about 400 ml, about 1 ml to about 500 ml, about 5 ml to about 10 ml, about 5 ml to about 25 ml, about 5 ml to about 50 ml, about 5 ml to about 100 ml, about 5 ml to about 150 ml, about 5 ml to about 200 ml, about 5 ml to about 250 ml, about 5 ml to about 300 ml, about 5 ml to about 400 ml, about 5 ml to about 500 ml, about 10 ml to about 25 ml, about 10 ml to about 50 ml, about 10 ml to about 100 ml, about 10 ml to about 150 ml, about 10 ml to about 200 ml, about 10 ml to about 250 ml, about 10 ml to about 300 ml, about 10 ml to about 400 ml, about 10 ml to about 500 ml, about 25 ml to about 50 ml, about 25 ml to about 100 ml, about 25 ml to about 150 ml, about 25 ml to about 200 ml, about 25 ml to about 250 ml, about 25 ml to about 300 ml, about 25 ml to about 400 ml, about 25 ml to about 500 ml, about 50 ml to about 100 ml, about 50 ml to about 150 ml, about 50 ml to about 200 ml, about 50 ml to about 250 ml, about 50 ml to about 300 ml, about 50 ml to about 400 ml, about 50 ml to about 500 ml, about 100 ml to about 150 ml, about 100 ml to about 200 ml, about 100 ml to about 250 ml, about 100 ml to about 300 ml, about 100 ml to about 400 ml, about 100 ml to about 500 ml, about 150 ml to about 200 ml, about 150 ml to about 250 ml, about 150 ml to about 300 ml, about 150 ml to about 400 ml, about 150 ml to about 500 ml, about 200 ml to about 250 ml, about 200 ml to about 300 ml, about 200 ml to about 400 ml, about 200 ml to about 500 ml, about 250 ml to about 300 ml, about 250 ml to about 400 ml, about 250 ml to about 500 ml, about 300 ml to about 400 ml, about 300 ml to about 500 ml, or about 400 ml to about 500 ml. In some embodiments, a volume of the spacer material that is injected is about 1 ml, about 5 ml, about 10 ml, about 25 ml, about 50 ml, about 100 ml, about 150 ml, about 200 ml, about 250 ml, about 300 ml, about 400 ml, or about 500 ml. In some embodiments, a volume of the spacer material that is injected is at least about 1 ml, about 5 ml, about 10 ml, about 25 ml, about 50 ml, about 100 ml, about 150 ml, about 200 ml, about 250 ml, about 300 ml, or about 400 ml. In some embodiments, a volume of the spacer material that is injected is at most about 5 ml, about 10 ml, about 25 ml, about 50 ml, about 100 ml, about 150 ml, about 200 ml, about 250 ml, about 300 ml, about 400 ml, or about 500 ml.

Administration may be performed in any suitable way, such as via injection from standard cannula and needles of appropriate sizes. The administration is performed where the radiative protection is desired, such as the chin, cheeks or elsewhere in the face or body.

The spacing material herein is injectable through standard needles used in medicine, such as 20 gauge or larger needles. Alternatively, the spacing material comprising hyaluronic acid can be injected using any of the following sized needles:

| | | Gauge | | | | | | | | | | |
|---|---|---|---|---|---|---|---|---|---|---|---|---|
| | | 15 | 16 | 17 | 18 | 19 | 20 | 21 | 22 | 23 | 24 | 25 |
| Needle | 0.5 | A1 | A2 | A3 | A4 | A5 | A6 | A7 | A8 | A9 | A10 | A11 |
| Length | 0.375 | B1 | B2 | B3 | B4 | B5 | B6 | B7 | B8 | B9 | B10 | B11 |
| (inch) | .75 | C1 | C2 | C3 | C4 | C5 | C6 | C7 | C8 | C9 | C10 | C11 |
| | 1 | D1 | D2 | D3 | D4 | D5 | D6 | D7 | D8 | D9 | D10 | D11 |
| | 1.25 | E1 | E2 | E3 | E4 | E5 | E6 | E7 | E8 | E9 | E10 | E11 |
| | 1.5 | F1 | F2 | F3 | F4 | F5 | F6 | F7 | F8 | F9 | F10 | F11 |
| | 2 | G1 | G2 | G3 | G4 | G5 | G6 | G7 | G8 | G9 | G10 | G11 |

In some embodiments, an interior surface of the needle comprises a protrusion, a mesh, a constriction, or any combination thereof. In some embodiments, injecting the spacing material past the protrusion, the mesh, the constriction, or any combination thereof produces a gas bubble in the spacing material. In some embodiments, the gas bubble is a microbubble. In some embodiments, the mesh has a mesh spacing of about 20 μm to about 300 μm. In some embodiments, a size of the mesh spacing determines a size of the microbubbles produced thereby.

### Fiducial Markers

CT imaging enhancement development can be used to leverage the stability of Hyaluronic acid for use as fiducial marker. Gold fiducial markers today can have too much artifact on MRI but perform well on CT.

### Image Enhancement

As polyethylene glycol, hyaluronic acid, and NASHA gels can image poorly on CT scans, MRI, and TRUS (transrectal ultrasound), the additives and compositions are configured to allow a clinician to inject the organ spacing materials herein at an accurate location, through real-time scanning feedback. Further, such scans can be used for radiation planning. HA CT imaging enhancement would be a very significant feature, sparing the need for a patient MRI, cost, and the CT/MRI fusion step to treatment plan. Additionally, for boost and Accelerated Partial Breast Irradiation (APBI) planning it is very important to have a clear identification of seroma to enable accurate target volume contouring and to initiate cone beam image guided radiotherapy. However, as seroma is not always visible on CT-simulation, many surgeries, such as those requiring full thickness closure at time of surgery, are difficult to plan and can be ineligible for such APBI procedures. While clip placement at time of surgery has been used to aid such contouring, they often form unreliable, whereas high-Z clip materials deform the contouring images and low-Z clip materials, such as tantalum, are not visible.

However, the image enhancement agents provided herein exhibit a Z value that is sufficiently to enable perfect visibility on CT and paramagnetic moment for visibility on MRI, but not too high to avoid the degradation of seroma imaging. Further optimal visibility and image quality is achieved by varying the volume of the injected image enhancement agents while maintaining minimal expansion of the volume to be treated.

In one embodiment, the visualization additive comprises Iodine which enhances CT imaging, but may have no benefit to MRI and TRUS imaging. Further, Iodine can be an allergen.

In some embodiments, the visualization additive comprises a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, ioversol, Gadolinium, gadolinium dimeglumine, gadopentetic acid carbon-coated zirconium beads, calcium hydroxylapatite, superparamagnetic iron oxide, or any combination thereof. In some embodiments, the superparamagnetic iron oxide additive is a superparamagnetic iron oxide nanoparticle. In some embodiments, a concentration of the visualization additive in the polyethylene glycol, the hyaluronic acid, or both is about 1 mg/ml to about 10 mg/ml. In some embodiments, a concentration of the visualization additive in the spacer material is about 0.1% to about 15%. In some embodiments, a concentration of the visualization additive in the spacer material is at least about 0.1%. In some embodiments, the visualization additive has an outer width of at least about 20 microns. In some embodiments, the visualization additive has a diameter of at least about 20 microns.

In some embodiments, the visualization additive comprises a gas. In some embodiments, the gas is air, nitrogen, helium, oxygen, or any combination thereof. In some embodiments, the gas forms a plurality of bubbles within the spacer material. In some embodiments, the bubbles are microbubbles. In some embodiments, the microbubbles have a size from about 1 μm to about 100 μm. In some embodiments, the gas is injected into the spacer material. In some embodiments, the gas is injected into the spacer material while the spacer material is under pressure. In some embodiments, the spacer material is agitated in an environment containing the gas to form the microbubbles. In some embodiments, the spacer material is pressurized and agitated in an environment containing the gas to form the microbubbles. In some embodiments, the microbubbles are formed before injection of the spacer material into a subject. In some embodiments, the microbubbles are formed during injection of the spacer material into a subject. In some embodiments, the microbubbles are formed during injection of the spacer material into a subject, wherein a geometry of needle forms the microbubbles. In some embodiments, the microbubbles are formed in-situ. In some embodiments, the cross-linked viscoelastic medium entraps and stabilizes the microbubbles.

### Spacer Material Dissolvent

In one example, the spacer material can comprise polyethylene glycol, wherein the dissolvent comprises water. In another example, the spacer material can comprise hyaluronic acid, wherein the dissolvent comprises hylauronidase. In another example, a dissolvant can be used to treat. In another example, a dissolvent can be used to treat melanoma, where spacing can be overly inflated temporarily to create greater distance for larger doses and then reversed for cosmetic purposes.

Method of Visualizing a Cavity, Method of Making

### Subcutaneous Spacer Materials

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising polyethylene glycol particles. The particle size and concentration of the polyethylene glycol within the spacer material can be tuned to exhibit a hardness, density, or both to enable consistent and uniform injection and cavity formation.

In some embodiments, the implant comprises particles of one or more viscoelastic media dispersed in a physiological salt buffer, a suitable physiological salt solvent, or both. In some embodiments, the implant further comprises other additives, such as local anesthetics, anti-inflammatory drugs, antibiotics and supportive medications (e.g. bone growth factors or cells). In some embodiments, there may also be included a viscoelastic medium which may be formed of same material as the particles or a different material than the particles. In some embodiments, the viscoelastic medium is not present as particles.

Viscoelastic media according to embodiments can herein include gels, dispersions, solutions, suspensions, slurries and mixtures thereof. In some embodiments, the medium is present as a dispersion of gel or gel-like particles. The viscoelastic medium provided herein can be more resistant to biodegradation in vivo than natural polyethylene glycol. The prolonged presence of the stable viscoelastic substance is advantageous for the patient, since the time between treatments is increased. The viscoelastic media herein can be biocompatible, sterile, and present as particles.

Advantageously, the viscoelastic media herein are stable within, but can be impermanent under, physiological conditions. In some embodiments, about 70% to about 90%, of the viscoelastic medium remains for at least two weeks in vivo. In some embodiments, at least 70%, of the viscoelastic medium remains for at about two weeks and two years in vivo. In some embodiments, at least 90%, of the viscoelastic medium remains for at about two weeks and two years in vivo. The viscoelastic medium can degrade automatically after five years or more in vivo.

Viscoelastic media include, without being limited thereto, polysaccharides and derivatives thereof. Suitable viscoelastic media include stabilized starch and derivatives thereof. Suitable viscoelastic media can also be selected from stabilized glycosaminoglycans and derivatives thereof, such as stabilized polyethylene glycol, stabilized chondroitin sulfate, stabilized heparin, and derivatives thereof. Suitable viscoelastic media also include stabilized dextran and derivatives thereof, such as dextranomer. In some embodiments, the dextronamer has a molecular weight of about 40 kDa to about 70 kDa. In some embodiments, the dextronamer has a molecular weight of at most about 70 kDa. In some embodiments, the dextronamer has a molecular weight of at least about 40 kDa. In some embodiments, the dextronamer is composed of microspheres. In some embodiments, the microspheres have a diameter of about 80 μm to about 250 μm. In some embodiments, the microspheres have a diameter of at least about 80 μm. In some embodiments, the microspheres have a diameter of at most about 250 μm. In some embodiments, the viscoelastic medium is cross-linked polyethylene glycol, or a derivatives thereof. An example of an viscoelastic medium is non-animal stabilized polyethylene glycol. One type of suitable cross-linked polyethylene glycol is obtainable by cross-linking of polyethylene glycol. The viscoelastic medium may also be a combination of two or more of the suitable viscoelastic media listed herein or otherwise known to the art. The viscoelastic medium may be of non-animal origin.

The size of the gel particles can depend upon the ionic strength of the buffer, the solution, carrier, or any combination thereof that is included in and/or surrounding the gel particles. As such, given particle sizes can assume physiological conditions, particularly isotonic conditions. In some embodiments, the gel particles contain and are dispersed in a physiological salt solution. In some embodiments, the gel particles are temporarily brought to different sizes by subjecting the gel particles to a solution of another tonicity. Particle sizes within the given ranges under physiological conditions when implanted subepidermally in the body or when subjected to a physiological, or isotonic, salt solution (i.e. a solution with the same tonicity as the relevant biological fluids, such as an isoosmotic with serum).

In some embodiments, the particles have a specific tuned size. The size of the particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size. Particle size may be determined in any suitable way, such as by laser diffraction, microscopy, or filtration, etc. In some embodiments, the specific shape of the gel particles is not critical. The size of a spherical particle can equal its diameter. The size may be measured as an average size, a median size, a maximum size, or a minimum size.

In some embodiments, the particles have a size in the range of from 1 to 2.5 mm, such as from 1.5 to 2 mm, in the presence of a physiological salt solution. In some embodiments, the particles have a size in the range of from 2.5 to 5 mm, such as from 3 to 4 mm, in the presence of a physiological salt solution. At least 50% (v/v) of the particles can have a size of at least about 1 mm. At least 50% (v/v) of the particles can have a size of about 1-5 mm in the presence of a physiological salt solution. In some embodiments, more than 70% (v/v) of the particles are within the given size limits under physiological conditions. In some embodiments, more than 90% (v/v) of the particles are within the given size limits under physiological conditions. Administration of the implant employing the method according to an embodiment herein prevents or diminishes migration and/or displacement of the implant, which comprises or consists of the 1-5 mm large particles under physiological conditions. Large particles can exhibit less in vitro migration and can be more easily removed. In some embodiments, the viscoelastic medium is not present as particles of a size smaller than 0.1 mm.

In some embodiments the particles have a size of about 0.1 mm to about 10 mm. In some embodiments the particles have a size of about 0.1 mm to about 0.2 mm, about 0.1 mm to about 0.5 mm, about 0.1 mm to about 1 mm, about 0.1 mm to about 1.5 mm, about 0.1 mm to about 2 mm, about 0.1 mm to about 3 mm, about 0.1 mm to about 4 mm, about 0.1 mm to about 5 mm, about 0.1 mm to about 6 mm, about 0.1 mm to about 8 mm, about 0.1 mm to about 10 mm, about 0.2 mm to about 0.5 mm, about 0.2 mm to about 1 mm, about 0.2 mm to about 1.5 mm, about 0.2 mm to about 2 mm, about 0.2 mm to about 3 mm, about 0.2 mm to about 4 mm, about 0.2 mm to about 5 mm, about 0.2 mm to about 6 mm, about 0.2 mm to about 8 mm, about 0.2 mm to about 10 mm, about 0.5 mm to about 1 mm, about 0.5 mm to about 1.5 mm, about 0.5 mm to about 2 mm, about 0.5 mm to about 3 mm, about 0.5 mm to about 4 mm, about 0.5 mm to about 5 mm, about 0.5 mm to about 6 mm, about 0.5 mm to about 8 mm, about 0.5 mm to about 10 mm, about 1 mm to about 1.5 mm, about 1 mm to about 2 mm, about 1 mm to about 3 mm, about 1 mm to about 4 mm, about 1 mm to about 5 mm, about 1 mm to about 6 mm, about 1 mm to about 8 mm, about 1 mm to about 10 mm, about 1.5 mm to about 2 mm, about 1.5 mm to about 3 mm, about 1.5 mm to about 4 mm, about 1.5 mm to about 5 mm, about 1.5 mm to about 6 mm, about 1.5 mm to about 8 mm, about 1.5 mm to about 10 mm, about 2 mm to about 3 mm, about 2 mm to about 4 mm, about 2 mm to about 5 mm, about 2 mm to about 6 mm, about 2 mm to about 8 mm, about 2 mm to about 10 mm, about 3 mm to about 4 mm, about 3 mm to about 5 mm, about 3 mm to about 6 mm, about 3 mm to about 8 mm, about 3 mm to about 10 mm, about 4 mm to about 5 mm, about 4 mm to about 6 mm, about 4 mm to about 8 mm, about 4 mm to about 10 mm, about 5 mm to about 6 mm, about 5 mm to about 8 mm, about 5 mm to about 10 mm, about 6 mm to about 8 mm, about 6 mm to about 10 mm, or about 8 mm to about 10 mm. In some embodiments the particles have a size of about 0.1 mm, about 0.2 mm, about 0.5 mm, about 1 mm, about 1.5 mm, about 2 mm, about 3 mm, about 4 mm, about 5 mm, about 6 mm, about 8 mm, or about 10 mm. In some embodiments the particles have a size of at least about 0.1 mm, about 0.2 mm, about 0.5 mm, about 1 mm, about 1.5 mm, about 2 mm, about 3 mm, about 4 mm, about 5 mm, about 6 mm, or about 8 mm. In some embodiments the particles have a size of at most about 0.2 mm, about 0.5 mm, about 1 mm, about 1.5 mm, about 2 mm, about 3 mm, about 4 mm, about 5 mm, about 6 mm, about 8 mm, or about 10 mm.

In some embodiments, the particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. Harder particles can be less viscoelastic and exhibit a longer half-life in vivo than softer particles. The particles herein should retain enough viscoelastic properties that they can be safely injected. In some embodiments, the implant comprises both soft gel particles and harder gel particles. The soft and hard gel particles may be made of the same or different viscoelastic media. The resulting mixture of gel particles combines desirable properties of softness/hardness for use in radiative protection and long durability in vivo.

### Methods of Forming a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising polyethylene glycol particles. The particle size and concentration of the polyethylene glycol within the spacer material can be tuned to exhibit a hardness, density, or both to enable consistent and uniform injection and cavity formation.

Provided herein are methods of forming a spacer material comprising forming an aqueous solution comprising: a water soluble cross-linkable polysaccharide; initiating a cross-linking of the polysaccharide in the presence of a polyfunctional cross-linking agent; sterically hindering the cross-linking reaction from terminating before gelation occurs to generate activated polysaccharide; and reintroducing the sterically unhindered conditions for the activated polysaccharide to continue the cross-linking thereof up to a viscoelastic gel. In some embodiments, the initial cross-linking reaction in the presence of a polyfunctional cross-linking agent can be performed at varying pH values, primarily depending on whether ether or ester reactions should be promoted.

The cross-linking agent can be any previously known cross-linking agent useful in connection with polysaccharides that are biocompatible. However, the cross-linking agent is comprises: aldehydes, epoxides, polyaziridyl compounds, glycidyl ethers, divinylsulfones, or any combination thereof. Glycidyl ethers represent an group, of which 1,4-butanediol diglycidyl ether can be advantageous. In some embodiments, the spacer material comprises a hydrogel comprising a glycosaminoglycan that is extracted from a natural source that is purified and derivatized. In some embodiments, the glycosaminoglycan is synthetically produced or synthesized by modified microorganisms such as bacteria. In some embodiments, the glycosaminoglycan is modified synthetically from a naturally soluble state to a partially soluble or water swellable or hydrogel state.

A suitable way of obtaining a desired particle size involves producing a gel made of cross-linked polyethylene glycol at a desired concentration and subjecting the gel to physical disruption, such as mincing, mashing or allowing the gel to pass through a filter with suitable particle size. The resulting gel particles are dispersed in a physiological salt solution, resulting in a gel dispersion or slurry with particles of desired size. The size of the particles can be achieved by producing a gel made of a viscoelastic medium at a desired concentration, and subjecting the gel to a physical disruption. The physical disruption can comprise: mincing, mashing filtering, or any combination thereof. The resulting gel particles can be dispersed in a physiological salt solution, resulting in a gel dispersion or slurry with particles of desired size.

In some embodiments, the particles have a specific tuned density, hardness or both. The gel particle density can be regulated by adjusting the concentration of the viscoelastic medium, the amount and type of cross-linking agent, or both. Harder particles can be achieved by increased concentration of the viscoelastic medium in the gel. By varying the polyethylene glycol concentrations to, for example, 20, 25, 40, 50 and 100 mg/ml gel particles of varying hardness can be obtained. Harder particles can be less viscoelastic and exhibit a longer half-life in vivo than softer particles. The particles herein should retain enough viscoelastic properties that they can be safely injected.

In some embodiments, the implant comprises both soft gel particles and harder gel particles.

The soft and hard gel particles may be made of the same or different viscoelastic media. The resulting mixture of gel particles combines desirable properties of softness/hardness for use in radiative protection and long durability in vivo. In one embodiment the soft gel particles comprise 15-22 mg/ml of the cross-linked polyethylene glycol, and the hard gel particles comprise 22-30 mg/ml of the cross-linked polyethylene glycol.

When the injectable medium is a polyethylene glycol medium, the polyethylene glycol concentration can be at least about 5 mg/ml. In some embodiments, the polyethylene glycol concentration is about 5 mg/ml to about 100 mg/ml. In some embodiments, the polyethylene glycol concentration is about 10 to about 50 mg/ml. In some embodiments, the polyethylene glycol concentration is about 20 mg/ml. The cross-linked polyethylene glycol can be present as particles or beads of any form.

In some embodiments, the concentration of the polyethylene glycol in the spacer material is about 1 mg/ml to about 100 mg/ml. In some embodiments, the concentration of the polyethylene glycol in the spacer material is about 1 mg/ml to about 5 mg/ml, about 1 mg/ml to about 10 mg/ml, about 1 mg/ml to about 15 mg/ml, about 1 mg/ml to about 20 mg/ml, about 1 mg/ml to about 25 mg/ml, about 1 mg/ml to about 30 mg/ml, about 1 mg/ml to about 40 mg/ml, about 1 mg/ml to about 50 mg/ml, about 1 mg/ml to about 60 mg/ml, about 1 mg/ml to about 80 mg/ml, about 1 mg/ml to about 100 mg/ml, about 5 mg/ml to about 10 mg/ml, about 5 mg/ml to about 15 mg/ml, about 5 mg/ml to about 20 mg/ml, about 5 mg/ml to about 25 mg/ml, about 5 mg/ml to about 30 mg/ml, about 5 mg/ml to about 40 mg/ml, about 5 mg/ml to about 50 mg/ml, about 5 mg/ml to about 60 mg/ml, about 5 mg/ml to about 80 mg/ml, about 5 mg/ml to about 100 mg/ml, about 10 mg/ml to about 15 mg/ml, about 10 mg/ml to about 20 mg/ml, about 10 mg/ml to about 25 mg/ml, about 10 mg/ml to about 30 mg/ml, about 10 mg/ml to about 40 mg/ml, about 10 mg/ml to about 50 mg/ml, about 10 mg/ml to about 60 mg/ml, about 10 mg/ml to about 80 mg/ml, about 10 mg/ml to about 100 mg/ml, about 15 mg/ml to about 20 mg/ml, about 15 mg/ml to about 25 mg/ml, about 15 mg/ml to about 30 mg/ml, about 15 mg/ml to about 40 mg/ml, about 15 mg/ml to about 50 mg/ml, about 15 mg/ml to about 60 mg/ml, about 15 mg/ml to about 80 mg/ml, about 15 mg/ml to about 100 mg/ml, about 20 mg/ml to about 25 mg/ml, about 20 mg/ml to about 30 mg/ml, about 20 mg/ml to about 40 mg/ml, about 20 mg/ml to about 50 mg/ml, about 20 mg/ml to about 60 mg/ml, about 20 mg/ml to about 80 mg/ml, about 20 mg/ml to about 100 mg/ml, about 25 mg/ml to about 30 mg/ml, about 25 mg/ml to about 40 mg/ml, about 25 mg/ml to about 50 mg/ml, about 25 mg/ml to about 60 mg/ml, about 25 mg/ml to about 80 mg/ml, about 25 mg/ml to about 100 mg/ml, about 30 mg/ml to about 40 mg/ml, about 30 mg/ml to about 50 mg/ml, about 30 mg/ml to about 60 mg/ml, about 30 mg/ml to about 80 mg/ml, about 30 mg/ml to about 100 mg/ml, about 40 mg/ml to about 50 mg/ml, about 40 mg/ml to about 60 mg/ml, about 40 mg/ml to about 80 mg/ml, about 40 mg/ml to about 100 mg/ml, about 50 mg/ml to about 60 mg/ml, about 50 mg/ml to about 80 mg/ml, about 50 mg/ml to about 100 mg/ml, about 60 mg/ml to about 80 mg/ml, about 60 mg/ml to about 100 mg/ml, or about 80 mg/ml to about 100 mg/ml. In some embodiments, the concentration of the polyethylene glycol in the spacer material is about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml. In some embodiments, the concentration of the polyethylene glycol in the spacer material is at least about 1 mg/ml, about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, or about 80 mg/ml. In some embodiments, the concentration of the polyethylene glycol in the spacer material is at most about 5 mg/ml, about 10 mg/ml, about 15 mg/ml, about 20 mg/ml, about 25 mg/ml, about 30 mg/ml, about 40 mg/ml, about 50 mg/ml, about 60 mg/ml, about 80 mg/ml, or about 100 mg/ml.

### Methods of Injecting a Spacer Material

The subcutaneous spacer materials herein are configured to form a cavity adjacent to prevent radiation or toxicity damage to organs proximal to or in contact with a treatment organ. The subcutaneous spacer materials herein may comprise a viscoelastic media comprising polyethylene glycol particles. The particle size and concentration of the polyethylene glycol within the spacer material can be tuned to exhibit a hardness, density, or both to enable consistent and uniform injection and cavity formation. Further, a specific needle size can be used to deliver the spacer material to its intended in vivo location based on the particle size, hardness, density, and concentration of the polyethylene glycol.

Provided herein is a method of injecting a spacing material. The spacing material can comprise a viscoelastic medium for therapeutic radiative protection in a mammal, including man. The spacing material can suitable for subepidermal administration at a site in said mammal where therapeutic soft tissue protection is required from radiation or other toxic sources. In particular, the particles are suitable for administration to tissues covered by publicly exposed skin, such as facial tissue, as the particles do not cause bruises or other discolorations. The particles herein are suitable for administration into deep subcutaneous or to submuscular/supraperiostal tissue, optionally in more than one layer. Deep subcutaneous or submuscular/supraperiostal administration can further prevent or diminished migration of the particles away from the desired site.

The spacing material can be administered by injection under the epidermis in any suitable way. By way of example, a dermal incision can be made with a scalpel or a sharp injection needle to facilitate transdermal insertion of a larger cannula for administration of the implant at the desired site.

The implant, consisting of particles of a viscoelastic medium and optionally other suitable ingredients, may be administered as a single aliquot or as layers of multiple aliquots. Optionally, the viscoelastic medium may be replaced, refilled or replenished by a subsequent injection of the same or another viscoelastic medium. The injected volume is determined by the size of the desired cavity.

In some embodiments, a volume of the spacer material that is injected is about 1 ml to about 500 ml. In some embodiments, a volume of the spacer material that is injected is about 1 ml to about 5 ml, about 1 ml to about 10 ml, about 1 ml to about 25 ml, about 1 ml to about 50 ml, about 1 ml to about 100 ml, about 1 ml to about 150 ml, about 1 ml to about 200 ml, about 1 ml to about 250 ml, about 1 ml to about 300 ml, about 1 ml to about 400 ml, about 1 ml to about 500 ml, about 5 ml to about 10 ml, about 5 ml to about 25 ml, about 5 ml to about 50 ml, about 5 ml to about 100 ml, about 5 ml to about 150 ml, about 5 ml to about 200 ml, about 5 ml to about 250 ml, about 5 ml to about 300 ml, about 5 ml to about 400 ml, about 5 ml to about 500 ml, about 10 ml to about 25 ml, about 10 ml to about 50 ml, about 10 ml to about 100 ml, about 10 ml to about 150 ml, about 10 ml to about 200 ml, about 10 ml to about 250 ml, about 10 ml to about 300 ml, about 10 ml to about 400 ml, about 10 ml to about 500 ml, about 25 ml to about 50 ml, about 25 ml to about 100 ml, about 25 ml to about 150 ml, about 25 ml to about 200 ml, about 25 ml to about 250 ml, about 25 ml to about 300 ml, about 25 ml to about 400 ml, about 25 ml to about 500 ml, about 50 ml to about 100 ml, about 50 ml to about 150 ml, about 50 ml to about 200 ml, about 50 ml to about 250 ml, about 50 ml to about 300 ml, about 50 ml to about 400 ml, about 50 ml to about 500 ml, about 100 ml to about 150 ml, about 100 ml to about 200 ml, about 100 ml to about 250 ml, about 100 ml to about 300 ml, about 100 ml to about 400 ml, about 100 ml to about 500 ml, about 150 ml to about 200 ml, about 150 ml to about 250 ml, about 150 ml to about 300 ml, about 150 ml to about 400 ml, about 150 ml to about 500 ml, about 200 ml to about 250 ml, about 200 ml to about 300 ml, about 200 ml to about 400 ml, about 200 ml to about 500 ml, about 250 ml to about 300 ml, about 250 ml to about 400 ml, about 250 ml to about 500 ml, about 300 ml to about 400 ml, about 300 ml to about 500 ml, or about 400 ml to about 500 ml. In some embodiments, a volume of the spacer material that is injected is about 1 ml, about 5 ml, about 10 ml, about 25 ml, about 50 ml, about 100 ml, about 150 ml, about 200 ml, about 250 ml, about 300 ml, about 400 ml, or about 500 ml. In some embodiments, a volume of the spacer material that is injected is at least about 1 ml, about 5 ml, about 10 ml, about 25 ml, about 50 ml, about 100 ml, about 150 ml, about 200 ml, about 250 ml, about 300 ml, or about 400 ml. In some embodiments, a volume of the spacer material that is injected is at most about 5 ml, about 10 ml, about 25 ml, about 50 ml, about 100 ml, about 150 ml, about 200 ml, about 250 ml, about 300 ml, about 400 ml, or about 500 ml.

Administration may be performed in any suitable way, such as via injection from standard cannula and needles of appropriate sizes. The administration is performed where the radiative protection is desired, such as the chin, cheeks or elsewhere in the face or body.

The spacing material herein is injectable through standard needles used in medicine, such as 20 gauge or larger needles. Alternatively the spacing material comprising polyethylene glycol can be injected using any of the following sized needles:

| | | Gauge | | | | | | | | | | |
|---|---|---|---|---|---|---|---|---|---|---|---|---|
| | | 15 | 16 | 17 | 18 | 19 | 20 | 21 | 22 | 23 | 24 | 25 |
| Needle | 0.5 | A1 | A2 | A3 | A4 | A5 | A6 | A7 | A8 | A9 | A10 | A11 |
| Length | 0.375 | B1 | B2 | B3 | B4 | B5 | B6 | B7 | B8 | B9 | B10 | B11 |
| (inch) | .75 | C1 | C2 | C3 | C4 | C5 | C6 | C7 | C8 | C9 | C10 | C11 |
| | 1 | D1 | D2 | D3 | D4 | D5 | D6 | D7 | D8 | D9 | D10 | D11 |
| | 1.25 | E1 | E2 | E3 | E4 | E5 | E6 | E7 | E8 | E9 | E10 | E11 |
| | 1.5 | F1 | F2 | F3 | F4 | F5 | F6 | F7 | F8 | F9 | F10 | F11 |
| | 2 | G1 | G2 | G3 | G4 | G5 | G6 | G7 | G8 | G9 | G10 | G11 |

In some embodiments, an interior surface of the needle comprises a protrusion, a mesh, a constriction, or any combination thereof. In some embodiments, injecting the spacing material past the protrusion, the mesh, the constriction, or any combination thereof produces a gas bubble in the spacing material. In some embodiments, the gas bubble is a microbubble. In some embodiments, the mesh has a mesh spacing of about 20 μm to about 300 μm. In some embodiments, a size of the mesh spacing determines a size of the microbubbles produced thereby.

### Fiducial Markers

CT imaging enhancement development can be used to leverage the stability of Polyethylene glycol for use as fiducial marker. Gold fiducial markers today can have too much artifact on MRI but perform well on CT.

### Image Enhancement

As polyethylene glycol, polyethylene glycol, and NASHA gels can image poorly on CT scans, MRI, and TRUS (transrectal ultrasound), the additives and compositions are configured to allow a clinician to inject the organ spacing materials herein at an accurate location, through real-time scanning feedback. Further, such scans can be used for radiation planning. HA CT imaging enhancement would be a very significant feature, sparing the need for a patient MRI, cost, and the CT/MRI fusion step to treatment plan. Additionally, for boost and Accelerated Partial Breast Irradiation (APBI) planning it is very important to have a clear identification of seroma to enable accurate target volume contouring and to initiate cone beam image guided radiotherapy. However, as seroma is not always visible on CT-simulation, many surgeries, such as those requiring full thickness closure at time of surgery, are difficult to plan and can be ineligible for such APBI procedures. While clip placement at time of surgery has been used to aid such contouring, they often form unreliable, whereas high-Z clip materials deform the contouring images and low-Z clip materials, such as tantalum, are not visible.

However, the image enhancement agents provided herein exhibit a Z value that is sufficiently to enable perfect visibility on CT and paramagnetic moment for visibility on MRI, but not too high to avoid the degradation of seroma imaging. Further optimal visibility and image quality is achieved by varying the volume of the injected image enhancement agents while maintaining minimal expansion of the volume to be treated.

In one embodiment, the visualization additive comprises Iodine which enhances CT imaging, but may have no benefit to MRI and TRUS imaging. Further, Iodine can be an allergen. In some embodiments, the visualization additives comprise a radiopaque compound selected from the group consisting of iohexol, metrizamide, iopamidol, 3,5-bis(acetylamino)-2,4,6-triiodobenzoic acid, meglumine diatrizoate, iopentol, iopromide, triiodobenzoic acid, erythrosine, ioversol, Gadolinium, gadolinium dimeglumine, gadopentetic acid carbon-coated zirconium beads, calcium hydroxylapatite, superparamagnetic iron oxide, or any combination thereof. In some embodiments, the superparamagnetic iron oxide additive is a superparamagnetic iron oxide nanoparticle. In some embodiments, a concentration of the visualization additive in the polyethylene glycol, the hyaluronic acid, or both is about 1 mg/ml to about 10 mg/ml. In some embodiments, the visualization additive comprises a gas. In some embodiments, the gas is air, nitrogen, helium, oxygen, or any combination thereof. In some embodiments, the gas forms a plurality of bubbles within the spacer material. In some embodiments, the microbubbles have a size from about 1 μm to about 100 μm. In some embodiments, a concentration of the visualization additive in the spacer material is about 0.1% to about 15%. In some embodiments, a concentration of the visualization additive in the spacer material is at least about 0.1%. In some embodiments, the visualization additive has an outer width of at least about 20 microns. In some embodiments, the visualization additive has a diameter of at least about 20 microns.

### Spacer Material Dissolvent

In some embodiments, the spacer materials herein can be configured to quickly dissolve into the body after a set period of time. Alternative, application of a dissolvent can initiate removal of the spacer material. Such a dissolvent can allow the spacer to be erased thus allowing for short term, temporary, larger spacing, to provide greater radioprotection, but which quickly reverses to maintain quality of life, cosmetic affect, or both thereafter.

In one example, the spacer material can comprise polyethylene glycol, wherein the dissolvent comprises hyaluronidase. In another example, a dissolvent can be used to treat melanoma, where spacing can be overly inflated temporarily to create greater distance for larger doses and then reversed for cosmetic purposes.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “radiative protection”, as used herein, refers to any type of volume augmentation of soft tissues, including, but not limited to, facial contouring (e.g. more pronounced cheeks or chin), correction of concave deformities (e.g. post-traumatic, HIV associated lipoatrophy) and correction of deep age-related facial folds. Thus, radiative protection may be used solely for cosmetic purposes or for medical purposes, such as following trauma or degenerative disease.

The term “degraded” implies that less than 20%, or less than 10%, of the medium remains in the body.

The term “soft tissue”, as used herein, refers to tissues that connect, support, or surround other structures and organs of the body. Soft tissue includes muscles, fibrous tissues and fat.

The terms “subepidermal administration” or “subcuticular administration”, as used herein, refer to administration beneath the epidermis of the skin, including administration into the dermis, subcutis or deeper, such as submuscularly or into the periosteum where applicable (in the vicinity of bone tissue.

As used herein, the term “therapeutic” involves any kind of preventive, alleviating or curative treatment.

By the term “super spacing,’ as used herein, is meant the dictionary definition of the terms “super” and “spacing.” Along with the definition of spacing that is extraordinary relative to traditional documentation of similar tissue spacing in a similarly situated part of a given subject.

As used herein, a physiological solution or isotonic solution is a solution having an osmolarity in the range of about 200-about 400 mOsm/l, about 250-about 350 mOsm/l, or about 300 mOsm/l. For practical purposes, this osmolarity can be achieved by preparation of a 0.9% (0.154 M) NaCl solution.

The term “implant”, as used herein, refers widely to any type of implanted or implantable foreign object or material. Implants also include objects or materials that are nearly identical to non-foreign objects or materials. The implant is not limited to any particular shape. The final shape of the implant in the body is decided by the skilled man from the purpose of the treatment.

By the term “viscoelastic medium”, as used herein, is meant a medium that exhibits a combination of viscous and elastic properties. Specifically, the viscoelastic medium is injectable through a 20 gauge or larger needle, such as a 10-20 gauge needle, by application of a pressure of 15-50 N. In particular, the medium, or an implant or a medicament comprising the medium, is suitable for subepidermal injection into a human in need thereof at a desired site.

By the term “stabilized”, as used herein, is meant any form of chemical stabilization that, under physiological conditions, renders the stabilized compound more stable to biodegradation that the parent compound. Without being limited thereto, stabilized compounds include cross-linked compounds and partially cross-linked compounds.

By the term “derivative” of a polysaccharide, as used herein, is meant any suitable derivative thereof, including cross-linked polysaccharides and substituted polysaccharides, such as sulfated polysaccharides.

### EXAMPLES

The following illustrative examples are representative of embodiments of the software applications, systems, and methods described herein and are not meant to be limiting in any way.

### Example 1—Spacer Injection Between the Head of Pancreas and the Duodenum

In one example, an absorbable hydrogel comprising hyaluronic acid microparticles was injected in the space between the Head of Pancreas (HOP) and the third portion of the duodenal loop using an 18-gauge needle. An endoscopic ultrasound (EUS) coupled to an ultrasound workstation was used to identify the duodenum and HOP interface, followed by hydrogel injection in this peripancreatic space using a 19-gauge fine needle aspiration needle in increments of 1 mL until the desired space was generated. The EUS scope was then adjusted (slightly advanced or retracted) around the target region to provide shape and conformity around the tumor to generate the desired space, with the total injection volume ranging from 1.0 mL to 27 mL.

A visible separation between the HOP and duodenum was created to confirm the location of the hydrogel and to measure the distance created between the duodenum and HOP. The mean distance of separation by hydrogel placement was measured by averaging the measured thickness of the gel on each CT slice on which gel was visualized on the post injection simulation CT scan obtained with a 2-mm slice thickness. The mean thickness of the spacer was 1.1 cm (0.9 cm-1.2 cm) and 0.9 cm (0.8 cm-1.1 cm) for EUS cadaveric specimens 1 and 2 on the post injection CT scans, respectively.

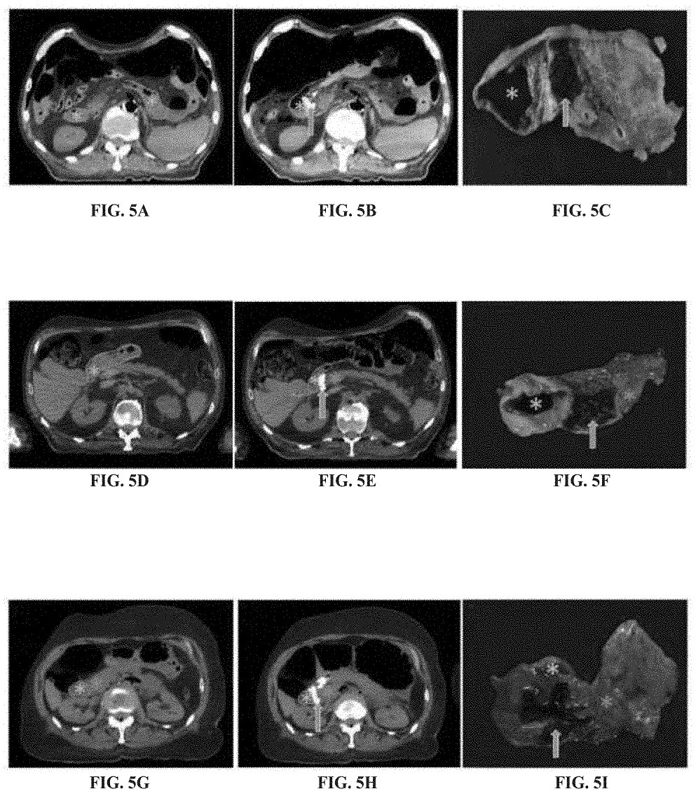

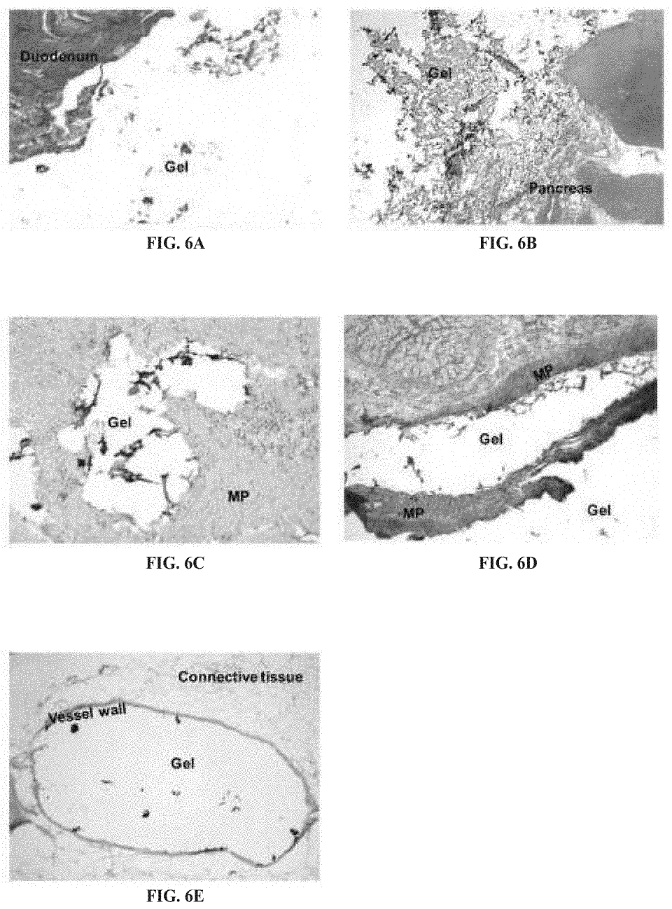

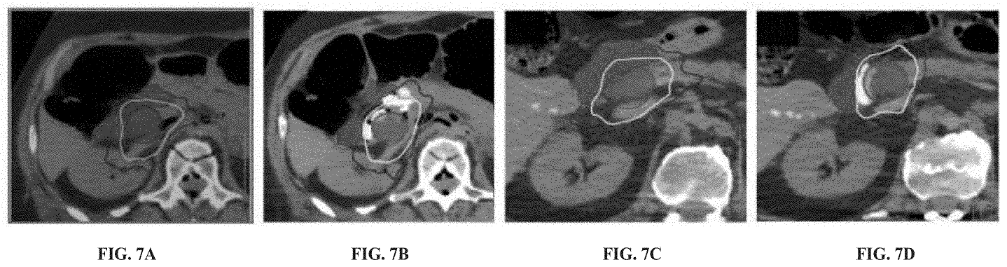

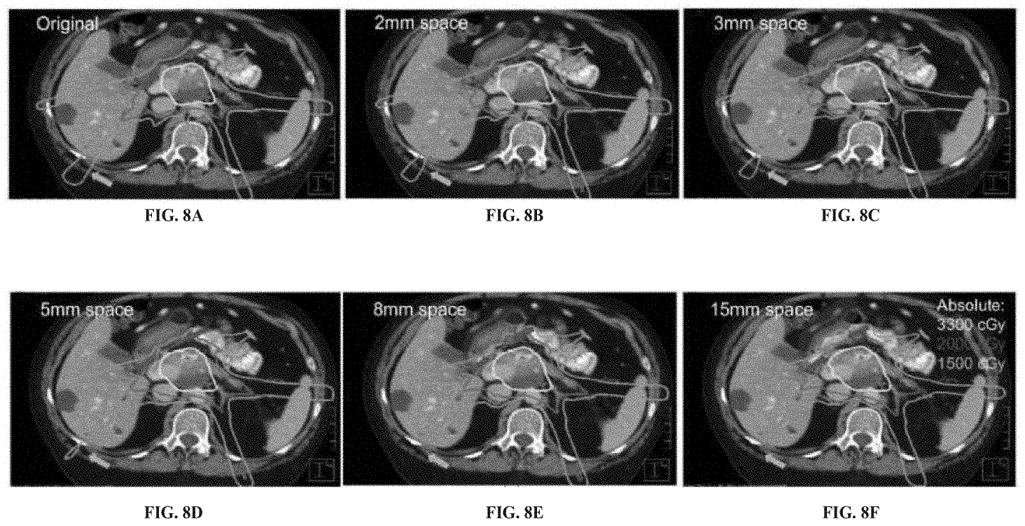

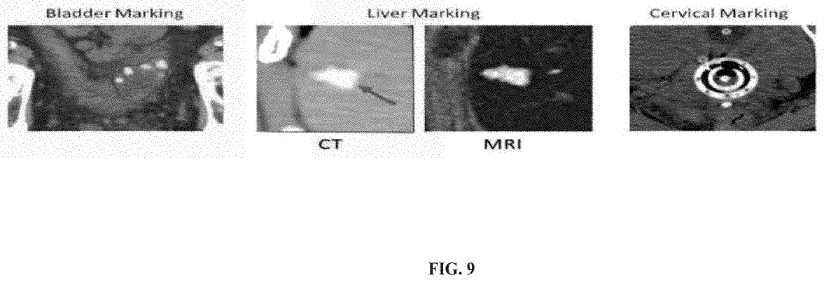

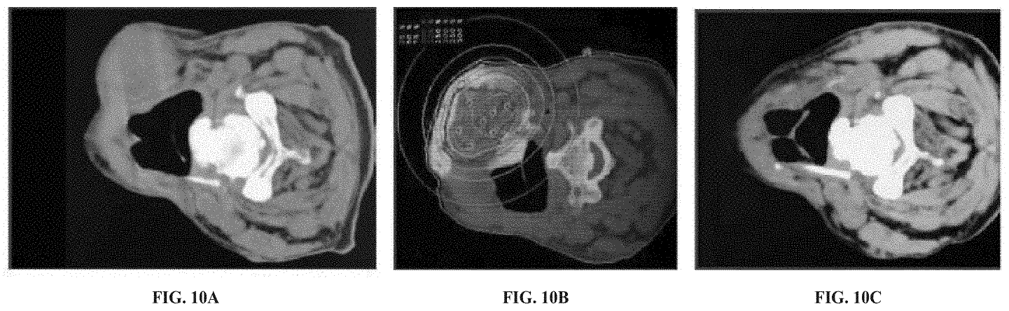

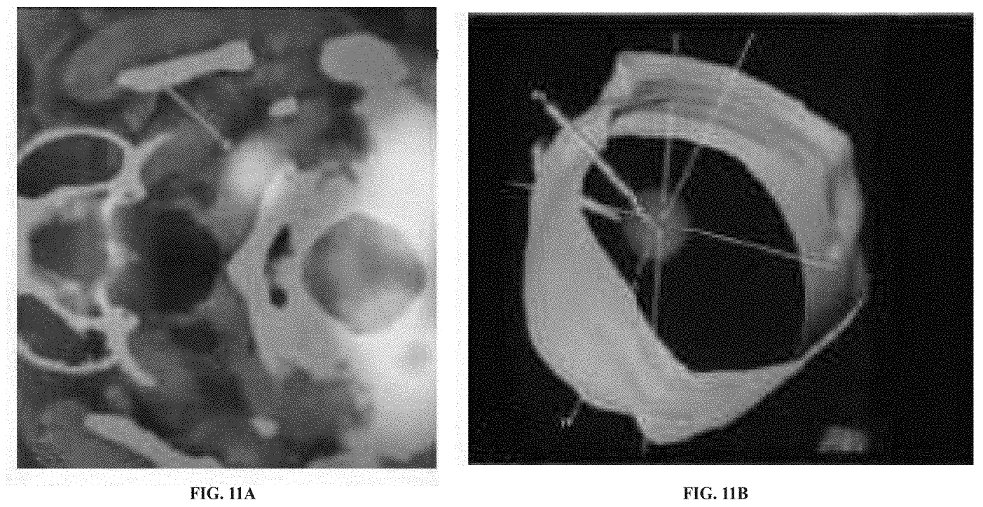

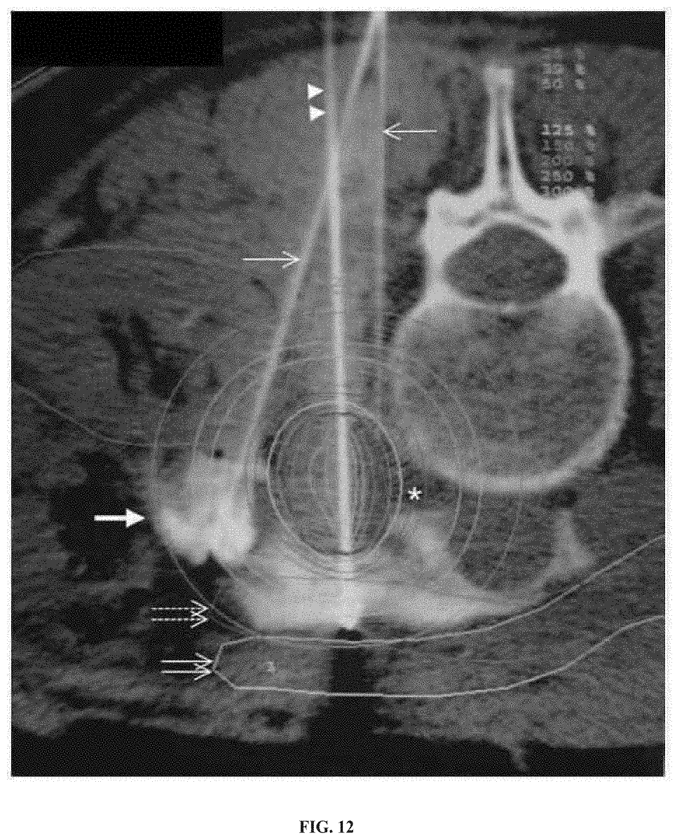

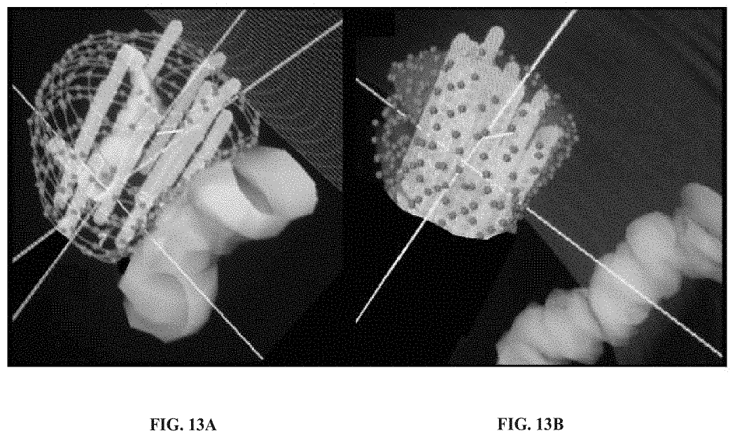

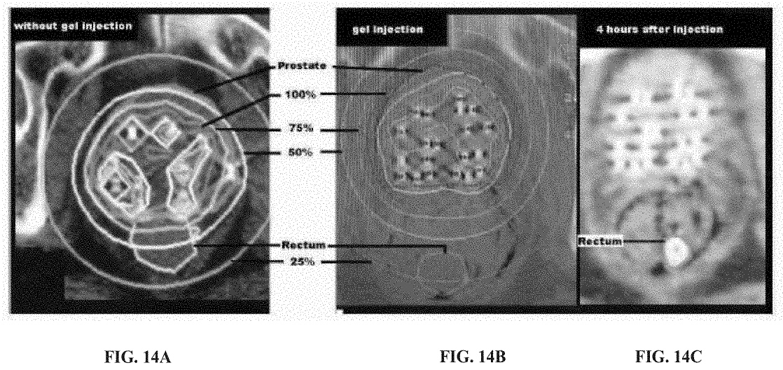

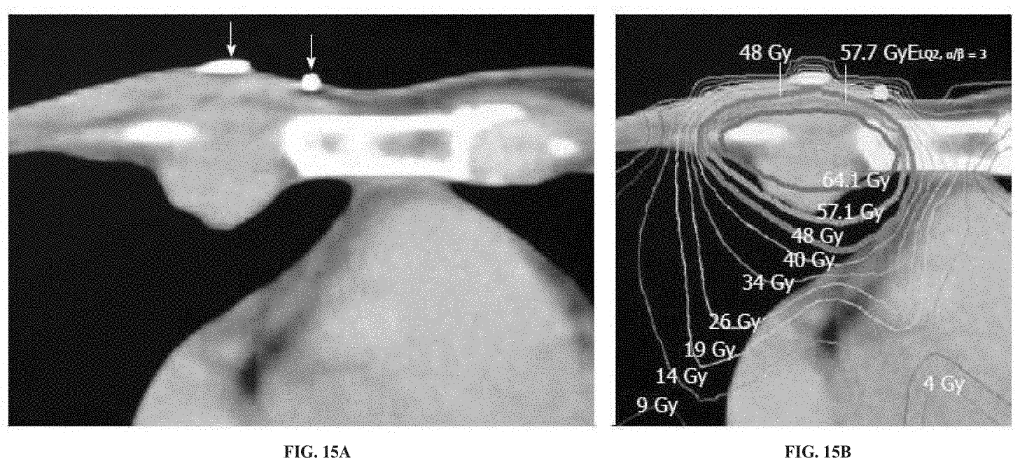

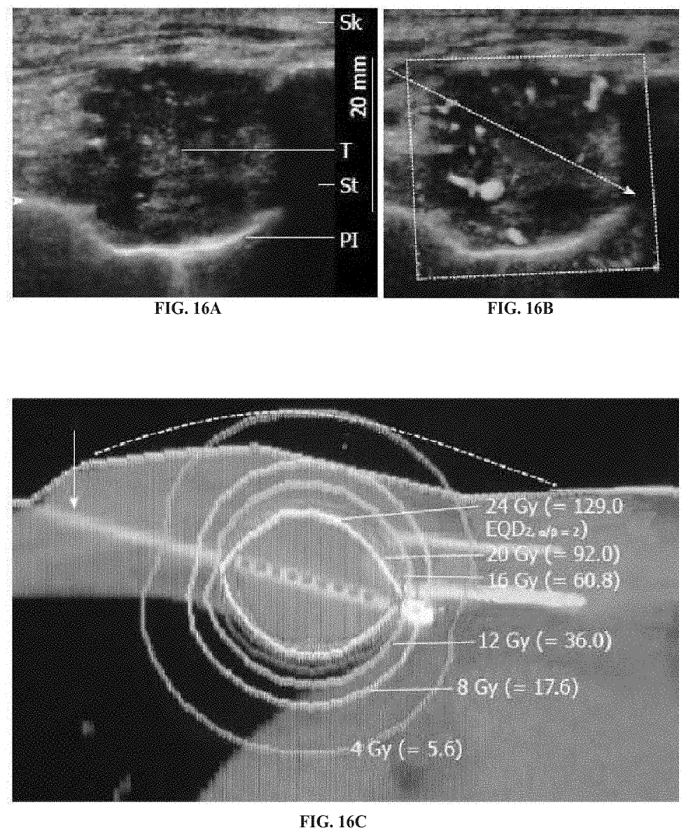

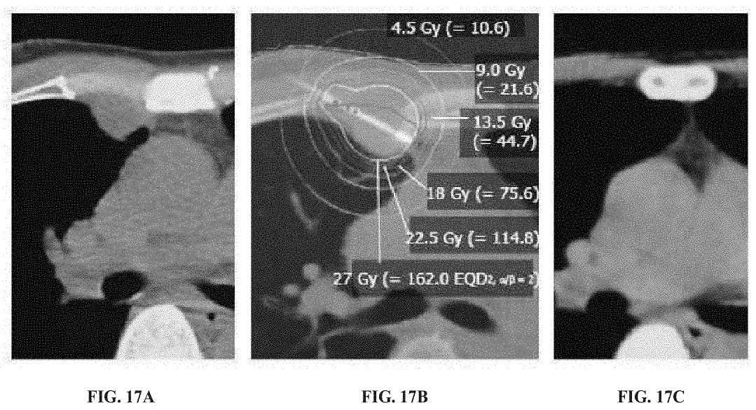

FIG. 5A is an exemplary image of a computed tomography scan before hydrogel spacer injection between the head of the pancreas and duodenum. FIG. 5B is an exemplary image of a computed tomography scan after hydrogel spacer injection between the head of the pancreas and duodenum. FIG. 5C is an exemplary image of a gross histologic specimen after hydrogel spacer injection between the head of the pancreas and duodenum. FIG. 5D is an exemplary image of a computed tomography scan before a laparotomy hydrogel spacer injection between the head of the pancreas and duodenum. FIG. 5E is an exemplary image of a computed tomography scan after a laparotomy hydrogel spacer injection between the head of the pancreas and duodenum. FIG. 5F is an exemplary image of a gross histologic specimen after a laparotomy hydrogel spacer injection between the head of the pancreas and duodenum. FIG. 5G is an exemplary image of a computed tomography scan before an endoscopically hydrogel spacer injection between the head of the pancreas and duodenum. FIG. 5H is an exemplary image of a computed tomography scan after an endoscopically hydrogel spacer injection between the head of the pancreas and duodenum. FIG. 5I is an exemplary image of a gross histologic specimen after an endoscopically hydrogel spacer injection between the head of the pancreas and duodenum. FIG. 6A is a first exemplary image of a formalin-fixed, paraffin-embedded section after hematoxylin and eosin staining. FIG. 6B is a second exemplary image of a formalin-fixed, paraffin-embedded section after hematoxylin and eosin staining. FIG. 6C is a first exemplary high magnification image of a formalin-fixed, paraffin-embedded section after hematoxylin and eosin staining. FIG. 6D is a third exemplary image of a formalin-fixed, paraffin-embedded section after hematoxylin and eosin staining. FIG. 6E is a second exemplary high magnification image of a formalin-fixed, paraffin-embedded section after hematoxylin and eosin staining. FIG. 7A is a first exemplary stereotactic body radiation therapy plan before hydrogel spacer placement. FIG. 7B is a first exemplary stereotactic body radiation therapy plan after hydrogel spacer placement. FIG. 7C is a second exemplary stereotactic body radiation therapy plan before hydrogel spacer placement. FIG. 7D is a second exemplary stereotactic body radiation therapy plan after hydrogel spacer placement. FIG. 8A is an exemplary baseline image of a computed tomography scan and stereotactic body radiation therapy plan of the duodenum. FIG. 8B is an exemplary image of a computed tomography scan and stereotactic body radiation therapy plan of the duodenum with a 2 mm spacing. FIG. 8C is an exemplary image of a computed tomography scan and stereotactic body radiation therapy plan of the duodenum with a 3 mm spacing. FIG. 8D is an exemplary image of a computed tomography scan and stereotactic body radiation therapy plan of the duodenum with a 5 mm spacing. FIG. 8E is an exemplary image of a computed tomography scan and stereotactic body radiation therapy plan of the duodenum with a 8 mm spacing. FIG. 8F is an exemplary image of a computed tomography scan and stereotactic body radiation therapy plan of the duodenum with a 15 mm spacing.

### Example 2—Subcutaneous Breast Spacer Injection

In one example, ultrasound-guided spacer injections of iodined polyethylene glycol (PEG) were performed to form a spacer thickness of greater than 5 mm. Pre and postinjection CT scans were used after defining a clinical target volume. Maximum dose to small skin volumes (D_{0.2 cc}) and existence of hotspots (isodose ≥90% on 1 cm2 of skin) were calculated as skin toxicity indicators. After removal of the breast, the spacer was injected directly under the skin to create a 5 mm extra space between skin and the superficial fascial layer of the breast by hydrodissection.

Intervention success was 90.9%. Hydrodissection was feasible in 63.6% of cases. Median system usability scale score was 82.5 for PEG (p<0.001). Mean D_{0.2 cc} was 80.8 Gy without spacer and 53.7 Gy with spacer (p<0.001). Skin hotspots were present in 40.9% without spacer but none with spacer (p<0.001).C

Success percentages of the spacer injection were high (91%) with a marker.

### Example 3—Subcutaneous Esophageal Spacer Injection

In one example, a gel was prepared as a viscous mixture of 10 ml of 1 mg/ml hyaluronic acid (HA), 0.8 ml of contrast media consisting of 300 mg iodine/ml. Following local anesthesia of subcutaneous tissue with lidocaine and under ultrasound and CT guidance, a 21-gauge needle was inserted at a puncture point lateral to the trachea at the level of the upper edge of the sternum and advanced to the location for gel injection. The needle penetrated the skin first; second, subcutaneous tissue (superficial cervical fascia, SCF) between both edges of the platysma in SCF; third, the relatively hard investing layer of deep cervical fascia (DCF) containing the suprasternal space (space of Burns) filled with adipose connective tissue and the transverse cervical vein, and adipose tissue below this fascia; and fourth, the pretracheal layer of DCF over the peritracheal space continuous with paraesophageal adipose tissue. To advance the needle safely, the trachea was shifted by about 5-10 mm manually to the right or left when necessary. Using this route, the needle passes medial to the sternohyoid and sternothyroid strap muscles to avoid the influence of muscle contraction on the needle. When the needle tip reached the predetermined injection point, the gel was injected to create a space, forcing the esophagus away from the target. The created space was confirmed by CT.

### Example 4—Reduction of Dose of Radiotherapy to Tissue Proximate to the Site of Radiotherapy to Pancreatic Cancer

Two patients are treated for pancreatic cancer by radiotherapy. One patient receives a PEG-based spacer injected using the technique described in Example 1. The injection technique from Example 1 is utilized to create a space between the site of radiotherapy and the duodenum between the range of 0.9 cm-1.2 cm. The dose of radiation in the tissue proximate to the site of radiotherapy is reduced by 40% in the patient whom received the spacer relative to the patient whom did not receive the spacer.

### Example 5—Reduction of Dose of Radiotherapy to Tissue Proximate to the Site of Radiotherapy to Esophageal Cancer

Two patients are treated for esophageal cancer by radiotherapy. One patient receives a PEG-based spacer injected using the technique described in Example 1. The injection technique from Example 4 is utilized to create a space between the site of radiotherapy and the duodenum between the range of 0.9 cm-1.2 cm. The dose of radiation in the tissue proximate to the site of radiotherapy is reduced by 20% in the patient whom received the spacer relative to the patient whom did not receive the spacer.

### Example 6—Erasable Hyaluronic Acid Spacer

The techniques described in Examples 4 and 5 are utilized. The patient is in rapid need of a reduction in the size of the spacer following radiotherapy due to the pressure exerted by the spacer onto the patient's esophagus. The caring physician injects a volume of about 1 ml to about 10 ml of hyaluronidase at about 1 U to about 100 U until the desired volume of the spacer is achieved.

### Example 7—Erasable PEG-Based Spacer

The techniques described in Examples 4 and 5 are utilized. The patient is in rapid need of a reduction in the size of the spacer following radiotherapy due to the pressure exerted by the spacer onto the patient's esophagus. The caring physician injects a volume of about 1 ml to about 50 ml of water until the desired volume of the spacer is achieved.

### Example 8—Shrinking Hyaluronic Acid Super Spacer

The injection technique of Example 4 is adapted to accommodate for a dual syringe. One syringe contains the desired volume of cross-linked hyaluronic acid particles while the opposite syringe contains a degradable nanoparticles containing hyaluronidase that are soluble in hyaluronic acid. The concentration of degradable nanoparticles contained in the syringe is determined by the determined length of radiotherapy. A larger spacing distance relative to the use of spacers without degradable nanoparticles containing hyaluronidase is achieved because the spacer shrinks before tissue damage due to great displacement of said tissue occurs or before undesired cosmetic changes to the anatomy of the subject occurs. This outcome is only made possible by the shrinking super spacer.

### Example 9—Use of Shrinking Hyaluronic Acid Super Spacer on a Subject Suffering from Melanoma on the Scalp

A subject has a melanoma tumor located his scalp. The tumor measures 0.4 cm×0.8 cm×0.2 cm. Radiotherapy is selected for as the desired treatment. The treating physician determines that the radiotherapy will require about 10 minutes. The treating physician desires to use a dose of radiotherapy that is greater than what is conventional due to the state of the tumor. The injection technique described in Examples 4 and 7 are adapted to a subcutaneous injection on the scalp. A volume between about 3 ml to about 6 ml is injected to space the tumor from the subject's brain. A specific proportion of this volume consists of the degradable nanoparticles containing hyaluronidase. This spaces the brain from the tumor by a distance that damages the connective tissue proximate to the skull and creates undesired cosmetic appearance. However, as soon as the spacer is injected in the space between the brain and the tumor, the spacer begins to shrink. Radiotherapy is performed and the subject's brain is exposed to about 20% less dose of radiotherapy compared to a subject that did not receive the spacer. The radiotherapy session concludes and the subject's scalp looks like the scalp of the subject that did not receive the spacer because the degradable nanoparticles have degraded the spacer.

### Example 10—Shrinking PEG-Based Super Spacer

The injection technique of Example 4 is adapted to accommodate for a dual syringe. One syringe contains the desired volume of PEG particles while the opposite syringe contains a degradable nanoparticles containing water that are soluble in hyaluronic acid. The concentration of degradable nanoparticles contained in the syringe is determined by the determined length of radiotherapy. A larger spacing distance relative to the use of spacers without degradable nanoparticles containing water is achieved because the spacer shrinks before tissue damage due to great displacement of said tissue occurs or before undesired cosmetic changes to the anatomy of the subject occurs. This outcome is only made possible by the shrinking super spacer.

### Example 11—Use of Shrinking PEG-Based Super Spacer on a Subject Suffering from Melanoma on the Scalp

A subject has a melanoma tumor located his scalp. The tumor measures 0.4 cm×0.8 cm×0.2 cm. Radiotherapy is selected for as the desired treatment. The treating physician determines that the radiotherapy will require about 10 minutes. The treating physician desires to use a dose of radiotherapy that is greater than what is conventional due to the state of the tumor. The injection technique described in Examples 4 and 7 are adapted to a subcutaneous injection on the scalp. A volume between about 3 ml to about 6 ml is injected to space the tumor from the subject's brain. A specific proportion of this volume consists of the degradable nanoparticles containing water. This spaces the brain from the tumor by a distance that damages the connective tissue proximate to the skull and creates a undesired cosmetic appearance. However, as soon as the spacer is injected in the space between the brain and the tumor, the spacer begins to shrink. Radiotherapy is performed and the subject's brain is exposed to about 20% less dose of radiotherapy compared to a subject that did not receive the spacer. The radiotherapy session concludes and the subject's scalp looks like the scalp of the subject that did not receive the spacer because the degradable nanoparticles have degraded the spacer.

### Example 12—Use of Improved Tissue Spacers for Interventional Oncology

7.4 g of poloxamer 407 and 0.2 g of polyethylene glycol-modified polylactic acid PLA-PEG, are added to 20 ml and 10 ml of pure water, and placed in a 25° C. for 3 days to completely dissolve the polymer. Then mixing the two and vortexing, results in a gel dispersion that is distilled off under reduced pressure and water. The spacer is then dried and sealed, and stored at 4° C. storage.

The hydrogel described above is inserted directly into a blood vessel that is directly couple to a tumor. The cancer is treated by the hydrogel blocking tumor blood supply, the tumor becomes ischemic, hypoxic and necrotic. Further, the cancer tissue necrosis continues to stimulate the body's immune system, it is possible to remove distant metastases (preferably in melanoma); delivering embolic agent(s) into the tumor with a chemotherapeutic agent mixed target artery feeding, both to block the blood supply, but also slows the release of chemotherapy drugs play a role in local chemotherapy, therefore, short-term efficacy of oncolytic outcomes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### Example 13—Iodine Incorporation into PEG

A PEG SG (with an SG count of 2.3 per molecule) containing an iodine core was synthesized. The PEG-I molecule was 6400 Daltons, of which iodine was 381 Daltons (5.9%). Thus, for example, with this iodine content, the percent solids of PEG-I in hydrogel that resulted in 0.1% and 0.2% iodine concentration in the resultant matrix was 1.68 and 3.36%. Table II shows how PEG-I concentrations can be manipulated to obtain a percentage iodine content, which in turn can be related to a CT number.

### Example +2—Preparing a Gadopentetate Dimeglumine with NASHA-Gel

A gel containing 5 mg/ml gadopentetate dimeglumine was prepared by weighing the gadopentetate dimeglumine and thoroughly mixing the gadopentetate dimeglumine with NASHA-gel by manual stirring. The resulting gel was centrifuged to remove air bubbles.

### Example +3—Adding Microbubbles to a Spacer Material During Injection

The spacing material described herein is drawn into a syringe. A needle is attached to the syringe wherein a portion of the interior surface of the needle contains a mesh structure. A dermal incision was made with a scalpel at the desired site and the spacing material was administered by injection under the epidermis, whereas microbubbles are formed within the spacing material as it passes through the mesh structure of the needle into the injection site.

### Example +4—Visualizing a Cavity

In another example, the spacer material herein combined with the visualization additives herein are injected into a treatment site of a patient. Thereafter, an MRI, CT, ultrasound, or any combination thereof is performed on the treatment site to determine that a cavity is formed by the spacer material between a treatment organ and a proximal organ.

1. A method of preventing or decreasing damage to a tissue proximate to a site of a radiotherapy in a subject undergoing the radiotherapy comprising injecting a bioabsorbable viscoelastic medium comprising a visualization additive at the site of the radiotherapy, wherein the visualization additive is present in an amount sufficient to generate contrast between the viscoelastic medium and the site of the radiotherapy.

2. (canceled)

3. The method of claim 1, wherein the viscoelastic medium comprises gel particles.

4. The method of claim 3, wherein the gel particles comprise hyaluronic acid or derivatives thereof.

5. The method of claim 1, wherein the injection comprises a volume of about 1 ml to about 50 ml.

6. (canceled)

7. The method of claim 4, wherein the concentration of hyaluronic acid is in the range of from about 5 mg/ml to about 100 mg/ml.

8. The method of claim 3, wherein the gel particles have a size range of about 0.2 mm to about 5 mm.

9. (canceled)

10. The method of claim 1, wherein migration of the viscoelastic medium is prevented or decreased.

11. The method of claim 1, wherein the visualization additive comprises a precious metal.

12. The method of claim 11, wherein the precious metal is gold.

13. The method of claim 1, wherein a dose of the radiotherapy contacting the tissue proximate to the site of radiotherapy is reduced by about 10% to about 80%.

14. The method of claim 1, wherein the site of the radiotherapy is selected from a group consisting of the subject's breast, head & neck, cervix, vagina, base of spine, skin, pancreas, liver, or lung.

15. (canceled)

16. The method of claim 1, wherein the volume of the viscoelastic medium at the site of radiotherapy is reduced by about 1% to about 95%.

17. The method of claim 1, wherein an administration of hyaluronidase occurs between about 0.1 hours to about 24 hours after the injection of the bioabsorbable viscoelastic medium.

18. The method of claim 1, further comprising imaging the site of the radiotherapy.

19-101. (canceled)

102. A formulation comprising a viscoelastic medium and a visualization additive present in an amount sufficient to generate contrast between the viscoelastic medium and mammalian tissue when imaged.

103. (canceled)

104. The formulation of claim 102, wherein the visualization additive comprises a precious metal.

105. The formulation of claim 104, wherein the precious metal is gold.

106. The formulation of claim 102, wherein the viscoelastic medium comprises gel particles.

107. The formulation of claim 106, wherein the gel particles comprise hyaluronic acid or derivatives thereof.

108. The formulation of claim 106, wherein the gel particles have a size range of about 0.2 mm to about 5 mm.
